SEQUENCE LISTING (31 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 288 aa, Macaca fascicularis
MQIPQAPWPVVWAVLQLGWRPGWFLESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQALVVGVVGGLLGSLVLLVWVLAVICSRAAQGTIEARRTGQPLKEDPSAVPVFSVDYGELDFQWREKTPEPPAPCVPEQTEYATIVFPSGLGTSSPARRGSADGPRSPRPLRPEDGHCSWPL

SEQ ID NO: 3 - protein, 288 aa, Macaca mulatta
MQIPQAPWPVVWAVLQLGWRPGWFLESPDRPWNPPTFSPALLLVTEGDNATFTCSFSNASESFVLNWYRMSPSNQTDKLAAFPEDRSQPGRDCRFRVTQLPNGRDFHMSVVRARRNDSGTYLCGAISLAPKAQIKESLRAELRVTERRAEVPTAHPSPSPRPAGQFQALVVGVVGGLLGSLVLLVWVLAVICSRAAQGTIEARRTGQPLKEDPSAVPVFSVDYGELDFQWREKTPEPPAPCVPEQTEYATIVFPSGLGTSSPARRGSADGPRSPRPLRPEDGHCSWPL

SEQ ID NO: 4 - protein, 288 aa, Mus musculus
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 5 - protein, 117 aa, Artificial Sequence
  source: /note=“Description of Artificial Sequence Synthetic polypeptide”
QVQLVQSGAEVKKPGASVKVSCKASGFRLTTNGISWVRQAPGQGLEWMGWISAGGGPTNYAQKLQGRVTMTTDTSTSTAYMELRSLRSDDTAVYYCAKGLYGTKDAWGQGTLVTVSS

SEQ ID NO: 6 - protein, 5 aa, Artificial Sequence
  source: /note=“Description of Artificial Sequence Synthetic peptide”
TNGIS

SEQ ID NO: 7 - protein, 17 aa, Artificial Sequence
  source: /note=“Description of Artificial Sequence Synthetic peptide”
WISAGGGPTNYAQKLQG

SEQ ID NO: 8 - protein, 8 aa, Artificial Sequence
  source: /note=“Description of Artificial Sequence Synthetic peptide”
GLYGTKDA

SEQ ID NO: 9 - protein, 110 aa, Artificial Sequence
  source: /note=“Description of Artificial Sequence Synthetic polypeptide”
QSVLTQPPSVSEVPGQRVTISCSGGGSNIGSNAVNWYQHFPGKAPKLLIYYNDLLPSGVSDRFSASKSGTSASLAISGLRSEDEADYYCAAWDDNLSAYVFATGTKVTVL

SEQ ID NO: 10 - protein, 13 aa, Artificial Sequence
  source: /note=“Description of Artificial Sequence Synthetic peptide”
SGGGSNIGSNAVN

SEQ ID NO: 11 - protein, 7 aa, Artificial Sequence
  source: /note=“Description of Artificial Sequence Synthetic peptide”
YNDLLPS

SEQ ID NO: 12 - protein, 11 aa, Artificial Sequence
  source: /note=“Description of Artificial Sequence Synthetic peptide”
AAWDDNLSAYV

SEQ ID NO: 13 - protein, 117 aa, Artificial Sequence; identical to SEQ ID NO: 5
  source: /note=“Description of Artificial Sequence Synthetic polypeptide”

SEQ ID NO: 14 - protein, 5 aa, Artificial Sequence
  source: /note=“Description of Artificial Sequence Synthetic peptide”
TNGIS

SEQ ID NO: 15 - protein, 17 aa, Artificial Sequence
  source: /note=“Description of Artificial Sequence Synthetic peptide”
WISAGGGPTNYAQKLQG

SEQ ID NO: 16 - protein, 8 aa, Artificial Sequence
  source: /note=“Description of Artificial Sequence Synthetic peptide”
GLYGTKDA

SEQ ID NO: 17 - protein, 110 aa, Artificial Sequence
  source: /note=“Description of Artificial Sequence Synthetic polypeptide”
QSVLTQPPSVSEVPGQRVTISCSGGGSNIGSNAVNWYQHFPGKAPKLLIYYNDLLPSGVSDRFSASKSGTSASLAISGLRSEDEADYYCAAWDDNLSAYVFGTGTKVTVL

SEQ ID NO: 18 - protein, 13 aa, Artificial Sequence
  source: /note=“Description of Artificial Sequence Synthetic peptide”
SGGGSNIGSNAVN

SEQ ID NO: 19 - protein, 7 aa, Artificial Sequence
  source: /note=“Description of Artificial Sequence Synthetic peptide”
YNDLLPS

SEQ ID NO: 20 - protein, 11 aa, Artificial Sequence
  source: /note=“Description of Artificial Sequence Synthetic peptide”
AAWDDNLSAYV

SEQ ID NO: 21 - protein, 120 aa, Artificial Sequence [sequence omitted]

SEQ ID NO: 22 - protein, 119 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKTSENLYFQ

SEQ ID NO: 23 - protein, 113 aa, Artificial Sequence [sequence omitted]

SEQ ID NO: 24 - protein, 107 aa, Artificial Sequence [sequence omitted]

SEQ ID NO: 25 - protein, 15 aa, Artificial Sequence
  source: /note=“Description of Artificial Sequence Synthetic peptide”
GGGGSGGGGSGGGGS

SEQ ID NO: 26 - protein, 19 aa, Mus sp.
MEWSWVFLFFLSVTTGVHS

SEQ ID NO: 27 - protein, 10 aa, Unknown
  source: /note=“Description of Unknown IgG1 upper hinge sequence”
EPKSCDKTHT

SEQ ID NO: 28 - protein, 9 aa, Unknown
  source: /note=“Description of Unknown lower hinge/CH2 sequence”
PAPELLGGP

SEQ ID NO: 29 - protein, 12 aa, Unknown
  source: /note=“Description of Unknown Fc region (CH2)”
SVFLFPPKPKDT

SEQ ID NO: 30 - protein, 23 aa, Unknown
  source: /note=“Description of Unknown hinge sequence”
EPKSCDKTHTCPPCPAPELLGGP

SEQ ID NO: 31 - protein, 30 aa, Artificial Sequence
  source: /note=“Description of Artificial Sequence Synthetic polypeptide”
  SITE (1)..(30): /note=“This sequence may encompass 1-6 ′Gly Gly Gly Gly Ser′ repeating units”
GGGGSGGGGSGGGGSGGGGSGGGGSGGGGS

(19) United States
(12) Patent Application Publication
(10) Pub. No.: US 2023/0235059 A1
(43) Pub. Date: Jul. 27, 2023

(54) ANTI-PD1 ANTIBODIES AND USES THEREOF

(71) Applicant: SORRENTO THERAPEUTICS, INC., San Diego, CA (US)

(72) Inventors: Heyue ZHOU, San Diego, CA (US); Priscilla TANG, San Diego, CA (US); Damien BRESSON, San Diego, CA (US); Barbara SWANSON, San Diego, CA (US); Alok SINGH, San Diego, CA (US); Lisa KERWIN, San Diego, CA (US)

(73) Assignee: SORRENTO THERAPEUTICS, INC., San Diego, CA (US)

(21) Appl. No.: 18/003,012

(22) PCT Filed: Jun. 25, 2021
(86) PCT No.: PCT/US2021/039188

§ 371 Date: Dec. 22, 2022

Related U.S. Application Data
(60) Provisional application No. 63/044,808, filed on Jun. 26, 2020.

(51) Int. Cl. C07K 16/28; A61P 35/00

(52) U.S. Cl. CPC C07K 16/2818; A61P 35/00; C07K 2317/21; C07K 2317/55; C07K 2317/622; C07K 2317/76; C07K 2317/33; C07K 2317/92; A61K 2039/505

(57) ABSTRACT

The present disclosure provides PD-1 binding proteins, particularly anti-PD-1 antibodies, or antigen-binding portions thereof, that specifically bind PD-1 and uses thereof. In one embodiment, the anti-PD-1 antibody comprises an antigen binding portion that binds a human PD-1 epitope or a non-human PD-1 epitope. Various aspects of the anti-PD-1 antibodies relate to antibody fragments, single-chain antibodies, pharmaceutical compositions, nucleic acids, recombinant expression vectors, host cells, and methods for preparing and using such anti-PD-1 antibodies. Methods for using the anti-PD-1 antibodies include in vitro and in vivo methods for binding PD-1, blocking interaction between PD-1 and PD-L1, detecting PD-1, and treating diseases associated with PD-L1 over-expression or PD-L1 detrimental expression.

This patent application claims priority to U.S. provisional application 63/044,808 filed Jun. 26, 2020, the contents of which is incorporated herein by reference in its entirety for all purposes.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### SEQUENCE LISTING

The instant application contains a Sequence Listing which has been submitted electronically in ASCII format and is hereby incorporated by reference in its entirety. Said ASCII copy, created on Jun. 22, 2021, is named 087735_0333_SL.txt and is 24,739 bytes in size.

### TECHNICAL FIELD

The present disclosure provides antigen binding proteins that bind specifically to PD-1 and nucleic acids that encode the antigen binding proteins, vectors comprising the nucleic acids, host cells harboring the vectors, and method of use thereof.

### BACKGROUND

Programmed cell death protein-1 (PD-1) is a type I membrane protein of 268 amino acids and is a member of the extended CD28/CTLA-4 family of T cell regulators PD-1 (The EMBO Journal (1992), vol. 11, issue 11, p. 3887-3895,). Human PD-1 cDNA is composed of the base sequence shown in EMBL/GenBank Acc. No. NM_005018 and mouse PD-1 cDNA is composed of the base sequence shown in Acc. No. NM_008798, and those expressions are observed when thymus cells differentiate from CD4−CD8− cell into CD4+CD8+ cell (International Immunology (1996), vol. 18, issue 5, p. 773-780., J. Experimental Med. (2000), vol. 191, issue 5, p. 891-898.). It is reported that PD-1 expression in periphery is observed in myeloid cells including T cells or B lymphocytes activated by stimulation from antigen receptors, or activated macrophages (International Immunology (1996), vol. 18, issue 5, p. 765-772).

PD-1 is a member of the CD28 family of receptors, which includes CD28, CTLA-4, ICOS, PD-1, and BTLA. The initial member of the family, CD28, was discovered by functional effect on augmenting T cell proliferation following the addition of monoclonal antibodies (Hutloff et al. (1999) Nature 397:263-266; Hansen et al. (1980) Immunogenics 10:247-260). Two cell surface glycoprotein ligands for PD-1 have been identified, PD-L1 and PDL-2, and have been shown to down-regulate T cell activation and cytokine secretion occur upon binding to PD-1 (Freeman et al. (2000) J. Exp. Med. 192:1027-34; Latchman et al. (2001) Nat. Immunol. 2:261-8; Carter et al. (2002) Eur. J. Immunol. 32:634-43; Ohigashi et al. (2005) Clin. Cancer Res. 11:2947-53). Both PD-L1 (B7-H1) and PD-L2 (B7-DC) are B7 homologs that bind to PD-1. Expression of PD-1 on the cell surface has also been shown to be upregulated through IFN-γ stimulation.

### SUMMARY

In one aspect, provided herein is a fully human anti-PD-1 antibody, or an antigen-binding fragment thereof, comprising a heavy chain and a light chain, the heavy chain and the light chain comprising: a) a heavy chain complementarity determining region 1 (CDR1) having the amino acid sequence of SEQ ID NO:6, a heavy chain CDR2 having the amino acid sequence of SEQ ID NO:7, a heavy chain CDR3 having the amino acid sequence of SEQ ID NO:8, a light chain CDR1 having the amino acid sequence of SEQ ID NO:10, a light chain CDR2 having the amino acid sequence of SEQ ID NO:11, and a light chain CDR3 having the amino acid sequence of SEQ ID NO:12 (e.g., herein called HPD-BB9); or b) a heavy chain complementarity determining region 1 (CDR1) having the amino acid sequence of SEQ ID NO:14, a heavy chain CDR2 having the amino acid sequence of SEQ ID NO:15, a heavy chain CDR3 having the amino acid sequence of SEQ ID NO:16, a light chain CDR1 having the amino acid sequence of SEQ ID NO:18, a light chain CDR2 having the amino acid sequence of SEQ ID NO:19, and a light chain CDR3 having the amino acid sequence of SEQ ID NO:20 (e.g., herein called HPD-BB9N).

In an aspect, provided herein is a fully human anti-PD-1 antibody, or an antigen-binding fragment thereof, comprising; a) a heavy chain and a light chain, the heavy chain comprising a heavy chain variable region having at least 95% sequence identity to the amino acid sequence of SEQ ID NO:5; and the light chain comprising a light chain variable region having at least 95% sequence identity to the amino acid sequence of SEQ ID NO:9; or b) a heavy chain and a light chain, the heavy chain comprising a heavy chain variable region having at least 95% sequence identity to the amino acid sequence of SEQ ID NO:13; and the light chain comprising a light chain variable region having at least 95% sequence identity to the amino acid sequence of SEQ ID NO:17.

In embodiments, the fully human anti-PD-1 antibody, or the antigen-binding fragment thereof, includes a) the heavy chain variable region and the light chain variable region which comprise the amino acid sequences of SEQ ID NOS:5 and 9 (e.g., herein called HPD-BB9); or b) the heavy chain variable region and the light chain variable region which comprise the amino acid sequences of SEQ ID NOS:13 and 17 (e.g., herein called HPD-BB9N).

In embodiments, the fully human anti-PD-1 antibody, or the antigen-binding fragment thereof, wherein the antigen-binding fragment is a Fab fragment comprising a variable domain region from a heavy chain and a variable domain region from a light chain, includes a) the variable domain region from the heavy chain comprises a sequence having at least 95% sequence identity to the amino acid sequence of SEQ ID NO:5, and wherein the variable domain region from the light chain comprises a sequence having at least 95% sequence identity to the amino acid sequence of SEQ ID NO:9; or b) the variable domain region from the heavy chain comprises a sequence having at least 95% sequence identity to the amino acid sequence of SEQ ID NO:13, and wherein the variable domain region from the light chain comprises a sequence having at least 95% sequence identity to the amino acid sequence of SEQ ID NO:17.

In embodiments, the Fab fragment includes a) the variable domain region from the heavy chain and the variable domain region from the light chain are SEQ ID NOS:5 and 9 (e.g., herein called HPD-BB9); or b) the variable domain region from the heavy chain and the variable domain region from the light chain are SEQ ID NOS:13 and 17 (e.g., herein called HPD-BB9N).

In embodiments, the fully human anti-PD-1 antibody, or the antigen-binding fragment thereof, wherein the antigen-binding fragment is a single chain antibody comprising variable domain region from a heavy chain and a variable domain region from a light chain joined together with a peptide linker, includes a) the variable domain region from the heavy chain comprises a sequence having at least 95% sequence identity to the amino acid sequence of SEQ ID NO:5, and wherein the variable domain region from the light chain comprises a sequence having at least 95% sequence identity to the amino acid sequence of SEQ ID NO:9; or b) the variable domain region from the heavy chain comprises a sequence having at least 95% sequence identity to the amino acid sequence of SEQ ID NO:13, and wherein the variable domain region from the light chain comprises a sequence having at least 95% sequence identity to the amino acid sequence of SEQ ID NO:17.

In embodiments, the single chain human anti-PD-1 antibody includes a) the variable domain region from the heavy chain and the variable domain region from the light chain are SEQ ID NOS:5 and 9 (e.g., herein called HPD-BB9); orb) the variable domain region from the heavy chain and the variable domain region from the light chain are SEQ ID NOS:13 and 17 (e.g., herein called HPD-BB9N).

In embodiments, any one of the disclosed fully human anti-PD-1 antibodies, or the antigen-binding fragment thereof, comprises an IgG1, IgG2, IgG3 or IgG4 antibody. In embodiments, any one of the disclosed fully human anti-PD-1 antibodies, or the antigen-binding fragment thereof, comprises an IgG1 or IgG4 isotype antibody.

In embodiments, any one of the disclosed fully human anti-PD-1 antibodies, or the antigen-binding fragment thereof, blocks binding of PD-1 protein to human PD-L1 protein. In embodiments, any one of the disclosed fully human anti-PD-1 antibodies, or the antigen-binding fragment thereof, binds to human PD-1 protein and cross-reacts with PD-1 protein from any one or any combination of cynomolgus monkey, rhesus monkey, mouse and/or dog. In embodiments, any one of the disclosed fully human anti-PD-1 antibodies, or the antigen-binding fragment thereof, binds to human PD-1 protein and does not cross-react with PD-1 protein from any one or any combination of cynomolgus monkey, rhesus monkey, mouse and/or dog.

In embodiments, any one of the disclosed fully human anti-PD-1 antibodies, or the antigen-binding fragment thereof, binds to human PD-1 protein expressed on the surface of human cells. In embodiments, any one of the disclosed fully human anti-PD-1 antibodies, or the antigen-binding fragment thereof, binds human PD-1 protein with a K_{D} of 10⁻⁷ M or less. In embodiments, any one of the disclosed fully human anti-PD-1 antibodies, or the antigen-binding fragment thereof, binds cynomolgus monkey PD-1 protein with a K_{D} of 10⁻⁷ M or less.

In embodiments, any one of the disclosed fully human anti-PD-1 antibodies, or the antigen-binding fragment thereof, binds rhesus monkey PD-1 protein with a K_{D} of 10⁻⁸ M or less. In embodiments, any one of the disclosed fully human anti-PD-1 antibodies, or the antigen-binding fragment thereof, binds mouse PD-1 protein with a K_{D} of 10⁻⁷ M or less.

In an aspect, provided herein is a pharmaceutical composition, comprising a pharmaceutically-acceptable excipient and any one of the disclosed human anti-PD-1 antibody or antigen-binding fragment. In an aspect, provided herein is a kit comprising any one of the disclosed human anti-PD-1 antibodies.

In an aspect, disclosed herein is a first nucleic acid that encodes a first polypeptide having the heavy chain variable region of any one of the disclosed human anti-PD-1 antibodies. In another aspect, disclosed herein is a second nucleic acid that encodes a second polypeptide having the heavy chain variable region of any one of the disclosed human anti-PD-1 antibodies. In another aspect, provided herein is a first nucleic acid that encodes a first polypeptide having the heavy chain variable region of any one of the disclosed human anti-PD-1 antibodies, and a second nucleic acid that encodes a second polypeptide having the light chain variable region of any one of the disclosed human anti-PD1 antibodies. In another aspect, provided herein is a nucleic acid that encodes a single chain antibody comprising a polypeptide having the heavy chain variable region of any one of the disclosed human anti-PD-1 antibodies, and encodes the light chain variable region of any one of the disclosed human anti-PD-1 antibodies.

In an aspect, provided herein is a first vector comprising the first nucleic acid. In an aspect, provided herein is a second vector comprising the second nucleic acid. In an aspect, provided herein is a (single) vector comprising the first and second nucleic acids. In an aspect, provided herein is a first vector comprising the first nucleic acid and a second vector comprising the second nucleic acid.

In an aspect, provided herein is a host cell harboring the first vector. In embodiments, the first vector, comprises a first expression vector, and the first host cell expresses the first polypeptide comprising the heavy chain variable region.

In an aspect, provided herein is a host cell harboring the second vector. In embodiments, the second vector, comprises a second expression vector, and the second host cell expresses the second polypeptide comprising the heavy chain variable region.

In an aspect, provided herein is a host cell harboring the (single) vector. In embodiments, the host cell includes the (single) vector which comprises an expression vector, wherein the host cell expresses the first polypeptide comprising the heavy chain variable region and expresses the second polypeptide comprising the light variable region.

In an aspect, provided herein is a host cell harboring the first vector and harboring the second vector. In embodiments, the host cell includes the first vector which comprises a first expression vector and the second vector comprises a second expression vector, and wherein the host cell expresses the first polypeptide comprising the heavy chain variable region and expresses the second polypeptide comprising the light variable region. In embodiments, the host cell includes the (single) vector which comprises an expression vector, and wherein the host cell expresses the single chain antibody comprising a polypeptide having the heavy chain variable region and the light variable region.

In an aspect, provided herein is a method for preparing a first polypeptide having an antibody heavy chain variable region, the method comprising: culturing a population of the host cell under conditions suitable for expressing the first polypeptide having the antibody heavy chain variable region. In embodiments, the method further comprising: recovering from the host cells the expressed first polypeptide having the antibody heavy chain variable region.

In an aspect, provided herein is a method for preparing a polypeptide having an antibody light chain variable region, the method comprising: culturing a population of the host cell under conditions suitable for expressing the second polypeptide having the antibody light chain variable region. In embodiments, the method further comprising: recovering from the host cells the expressed second polypeptide having the antibody light chain variable region.

In an aspect, provided herein is a method for preparing a first polypeptide having the antibody heavy chain variable region and a second polypeptide having the antibody light chain variable region, the method comprising: culturing a population of the host cell under conditions suitable for expressing the first polypeptide having the antibody heavy chain variable region and the second polypeptide having the antibody light chain variable region. In embodiments, the method further comprising: recovering from the host cells the expressed first polypeptide having the antibody heavy chain variable region and the expressed second polypeptide having the antibody light chain variable region.

In an aspect, provided herein is a method for preparing a single chain antibody having a heavy chain variable region and a light chain variable region, the method comprising: culturing a population of the host cell under conditions suitable for expressing polypeptide comprising the heavy chain variable region and the light chain variable region. In embodiments, the method further comprising: recovering from the host cells the expressed polypeptide comprising the heavy chain variable region and the light chain variable region.

In an aspect, provided herein is a method (e.g., in vitro or in vivo method) for blocking interaction between a PD-1-expressing cell and a PD-L1-expressing cell comprising: contacting any of the disclosed anti-PD1 antibodies with a PD-1-expressing cell and a PD-L1-expressing cell, under conditions suitable for binding between the anti-PD1 antibody and the PD-1-expressing cells and for blocking between the PD-1-expressing cell and the PD-L1-expressing cell. In embodiments, the PD-1-expressing cell comprises a T cell. In embodiments, the PD-L1-expressing cell comprises a tumor cell. In embodiments, the blocking of the interaction between the PD-1-expressing cell (e.g., T cell) and the PD-L1-expressing cell (e.g., tumor) by the anti-PD1 antibody blocks activation of a PD-1 receptor on the PD-1-expressing cell. In embodiments, the blocking of the interaction between the PD-1-expressing cell (e.g., T cell) and the PD-L1-expressing cell (e.g., tumor) by the anti-PD1 antibody causes activation of the PD-1-expressing cell (e.g., activation of the T cell).

In an aspect, provided herein is a method for treating a subject having a disease associated with PD-L1 over-expression or PD-L1 detrimental expression, the method comprising: administering to the subject an effective amount of a therapeutic composition comprising any one of the disclosed human anti-PD-1 antibodies. In embodiments, the disease associated with PD-L1 over-expression or PD-L1 detrimental expression is selected from a group consisting of cancer of the lung (including non-small cell lung and small cell lung cancers), prostate, breast, ovary, head and neck, thyroid, parathyroid gland, adrenal gland, bladder, intestine, skin, colorectal, anus, rectum, pancreas, leiomyoma, brain, glioma, glioblastoma, esophagus, liver, kidney, stomach, colon, cervix, uterus, fallopian tubes, endometrium, vulva, larynx, vagina, bone, nasal cavity, paranasal sinus, nasopharynx, oral cavity, oropharynx, larynx, hypolarynx, salivary glands, ureter, urethra, penis and testis.

### DESCRIPTION OF THE DRAWINGS

FIG. 1A shows an SPR sensorgram of binding kinetics of antibodies HPD-BB9, Keytruda and Opdivo, to human PD-1 antigen, with their respective binding affinity K_{D} values.

FIG. 1B shows an SPR sensorgram of binding kinetics of antibodies HPD-BB9, Keytruda and Opdivo, to cynomolgus monkey PD-1 antigen, with their respective binding affinity K_{D} values.

FIG. 1C shows an SPR sensorgram of binding kinetics of antibodies HPD-BB9, Keytruda and Opdivo, to rhesus monkey PD-1 antigen, with their respective binding affinity K_{D} values.

FIG. 1D shows an SPR sensorgram of binding kinetics of antibody HPD-BB9 to mouse PD-1 antigen, with its binding affinity K_{D} values.

FIG. 1E shows a table that summarizes binding kinetics obtained from SPR data, from FIGS. 1A-D, of antibodies HPD-BB9, Keytruda and Opdivo, with PD-1 antigen from different species.

FIG. 2A shows a graph of a cell binding assay of antibodies HDP-BB9, Keytruda and a control IgG4 isotype, binding to Raji cells that are not engineered to express human PD-1.

FIG. 2B shows a graph of a cell binding assay of antibodies HDP-BB9, Keytruda and a control IgG4 isotype, binding to Raji cells engineered to express human PD-1 antigen.

FIG. 3 shows histograms of flow cytometry data of binding between antibodies HDP-BB9, Keytruda or control secondary antibody, to PBMCs from human or dog.

FIG. 4 shows a bar graph comparing the level of interferon gamma (IFNγ) release generated from a mixed lymphocyte reaction (MRL) assay, comparing antibodies HDP-BB9, Keytruda, Opdivo and a control IgG4 isotype.

FIG. 5A shows a bar graph comparing the level of interferon gamma (IFNγ) release generated from a first experiment of a three-way mixed lymphocyte reaction (MLR) assay, comparing antibodies HDP-BB9, Keytruda, Opdivo and a control IgG4 isotype.

FIG. 5B shows a bar graph comparing the level of interferon gamma (IFNγ) release generated from a second experiment of a three-way mixed lymphocyte reaction (MLR) assay, comparing antibodies HDP-BB9, Keytruda, Opdivo and a control IgG4 isotype.

FIG. 6 shows the amino acid sequences of PD-1 antigens from human, cynomolgus monkey, rhesus monkey and mouse.

FIG. 7 shows the amino acid sequences of anti-PD1 antibody HDP-BB9, including the heavy chain variable region, and heavy chain CDRs 1, 2 and 3, and the light chain variable region, and light chain CDRs 1, 2 and 3. The CDR regions in the heavy and light chains are underlined.

FIG. 8 shows the amino acid sequences of anti-PD1 antibody HDP-BB9N, including the heavy chain variable region, and heavy chain CDRs 1, 2 and 3, and the light chain variable region, and light chain CDRs 1, 2 and 3. The CDR regions in the heavy and light chains are underlined.

FIG. 9 shows the amino acid sequences of anti-PD1 antibody Keytruda including the heavy chain variable region and the light chain variable region, and the amino acid sequences of anti-PD1 antibody Opdivo including the heavy chain variable region and the light chain variable region.

FIG. 10 shows a graph of dose-dependent response of the blocking of PD1/PD-L1 interaction using human anti-PD1 clones HPD-BB9 and KEYTRUDA and a negative control antibody (Isotype IgG4).

FIG. 11 shows the effect of human anti-PD1 clone HPD-BB9 on bladder tumor growth in MB-49 syngeneic tumor model. FIG. 11A shows the effect of 5 and 15 mg/kg of HPD-BB9 clone and 15 mg/kg of isotype control on the tumor volume of each individual mouse, measured over 24 days. FIG. 11B shows the effect of 5 and 15 mg/kg of HPD-BB9 clone and 15 mg/kg of isotype control on the tumor volume—averaged for the 10 mice, measured over 24 days. FIG. 11C shows percent tumor growth inhibition by HPD-BB9 clone (TGI=(1-[mean HPD-BB9/mean Isotype])×100) calculated at the end of the study day 24 post tumor cell implantation.

FIG. 12 shows the effect of human anti-PD1 clone HPD-BB9 and a negative (isotype) control IgG4 on body weight of each mouse with MB-49 syngeneic tumor model.

### DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules and mature molecule. Precursor molecules include those that have not yet been subjected to cleavage, for example cleavage by a secretory signal peptide or by non-enzymatic cleavage at certain amino acid residue. Polypeptides in include mature molecules that have undergone cleavage. These terms encompass native proteins, recombinant proteins and artificial proteins, protein fragments and polypeptide analogs (such as muteins, variants, chimeric proteins and fusion proteins) of a protein sequence as well as post-translationally, or otherwise covalently or non-covalently, modified proteins. Polypeptides comprising amino acid sequences of binding proteins that bind PD-1 (e.g., anti-PD-1 antibodies or antigen-binding portions thereof) prepared using recombinant procedures are described herein.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof. In one embodiment, nucleic acids comprise a one type of polynucleotides or a mixture of two or more different types of polynucleotides. Nucleic acids encoding anti-PD-1 antibodies or antigen-binding portions thereof, are described herein.

The term “recover” or “recovery” or “recovering”, and other related terms, refers to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide sequence (e.g., leader peptide sequence) which mediates secretion of the expressed protein. The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiments to protein or polynucleotides that are substantially free of other molecules of the same species, for example other protein or polynucleotides having different amino acid or nucleotide sequences, respectively. The purity or homogeneity of the desired molecule can be assayed using techniques well known in the art, including low resolution methods such as gel electrophoresis and high resolution methods such as HPLC or mass spectrometry. In one embodiment, any of the anti-PD-1 antibodies or antigen binding protein thereof are isolated.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “leader sequence” or “leader peptide” or “peptide signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length. A leader sequence can direct transport of a precursor polypeptide from the cytosol to the endoplasmic reticulum. In one embodiment, a leader sequence includes signal sequences comprising CD8α, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence MEWSWVFLFFLSVTTGVHS (SEQ ID NO: 26).

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins that bind PD-1 are described herein.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having immunoglobulin-like properties that bind specifically to PD-1 are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the segments FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5th Ed., US Dept. of Health and Human Services, PHS, NIH, NIH Publication no. 91-3242, 1991 (e.g., “Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Anti-PD-1 antibodies are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Antigen binding domains from anti-PD-1 antibodies are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ or less, or 10⁻¹² or less. Anti-PD-1 antibodies that specifically bind PD-1 are described herein.

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. Anti-PD-1 antibodies, and antigen binding proteins thereof, that bind an epitope of a PD-1 polypeptide are described herein.

With respect to antibodies, the term “antagonist” and “antagonistic” refers to a blocking antibody that binds its cognate target antigen and inhibits or reduces the biological activity of the bound antigen. The term “agonist” or “agonistic” refers to an antibody that binds its cognate target antigen in a manner that mimics the binding of the physiological ligand which causes antibody-mediated downstream signaling.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. Antigen-binding fragments of anti-PD-1 antibodies are described herein.

The terms “Fab”, “Fab fragment” and other related terms refers to a monovalent fragment comprising a variable light chain region (V_{L}), constant light chain region (C_{L}), variable heavy chain region (V_{II}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or VL domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). Fab fragments comprising antigen binding portions from anti-PD-1 antibodies are described herein.

A single-chain antibody (scFv) is an antibody in which a V_{L} and a V_{H} region are joined via a linker (e.g., a synthetic sequence of amino acid residues) to form a continuous protein chain. In one embodiment, the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). Single chain antibodies comprising antigen binding portions from anti-PD-1 antibodies are described herein.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the anti-PD1 antibodies described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Fully human anti-PD-1 antibodies and antigen binding proteins thereof are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the anti-PD-1 antibodies described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising full-length heavy chains and full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In one embodiment, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region can be derived from is a hinge region of a naturally-occurring protein, such as a CD8 hinge region or a fragment thereof, a CD8a hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In one embodiment, the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT (SEQ ID NO: 27). In one embodiment, the hinge region comprises an IgG1 core hinge sequence CPXC, wherein X is P, R or S. In one embodiment, the hinge region comprises a lower hinge/CH2 sequence PAPELLGGP (SEQ ID NO: 28). In one embodiment, the hinge is joined to an Fc region (CH2) having the amino acid sequence SVFLFPPKPKDT (SEQ ID NO: 29). In one embodiment, the hinge region includes the amino acid sequence of an upper, core and lower hinge and comprises EPKSCDKTHTCPPCPAP ELLGGP (SEQ ID NO: 30).

In one embodiment, the hinge region comprises one, two, three or more cysteines that can form at least one, two, three or more interchain disulfide bonds.

The term “Fe” or “Fc region” as used herein refers to the portion of an antibody heavy chain constant region beginning in or after the hinge region and ending at the C-terminus of the heavy chain. The Fc region comprises at least a portion of the CH2 and CH3 regions and may, or may not, include a portion of the hinge region. An Fc domain can bind Fc cell surface receptors and some proteins of the immune complement system. An Fc region can bind a complement component C1q. An Fc domain exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain can bind an Fc receptor, including FcγRI (e.g., CD64), FcγRII (e.g, CD32) and/or FcγRIII (e.g., CD16a). In one embodiment, the Fc region can include a mutation that increases or decreases any one or any combination of these functions. In one embodiment, the Fc domain comprises a LALA mutation (e.g., equivalent to L234A, L235A according to Kabat numbering) which reduces effector function. In one embodiment, the Fc domain comprises a LALA-PG mutation (e.g., equivalent to L234A, L235A, P329G according to Kabat numbering) which reduces effector function. In one embodiment, the Fc domain mediates serum half-life of the protein complex, and a mutation in the Fc domain can increase or decrease the serum half-life of the protein complex. In one embodiment, the Fc domain affects thermal stability of the protein complex, and mutation in the Fc domain can increase or decrease the thermal stability of the protein complex.

The term “labeled” or related terms as used herein with respect to a polypeptide refers to joinder antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-PD-1 antibodies described herein can be unlabeled or can be joined to a detectable label or moiety.

The term “labeled” or related terms as used herein with respect to a polypeptide refers to joinder thereof to a detectable label or moiety for detection. Exemplary detectable labels or moieties include radioactive, colorimetric, antigenic, enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-PD-1 antibodies described herein or antigen-binding portions thereof that described herein can be unlabeled or can be joined to a detectable label or detectable moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test antibody may be similar but not necessarily identical to any of the amino acid sequences of the polypeptides that make up any of the anti-PD-1 antibodies, or antigen binding protein thereof, described herein. The similarities between the test antibody and the polypeptides can be at least 95%, or at or at least 96% identical, or at least 97% identical, or at least 98% identical, or at least 99% identical, to any of the polypeptides that make up any of the anti-PD-1 antibodies, or antigen binding protein thereof, described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise at least a portion of any of the anti-PD-1 antibodies described herein.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been introduced with exogenous nucleic acid (transgene). The host cell includes the primary subject cell and its progeny. Exogenous nucleic acids encoding at least a portion of any of the anti-PD-1 antibodies described herein can be introduced into a host cell. Expression vectors comprising at least a portion of any of the anti-PD-1 antibodies described herein can be introduced into a host cell, and the host cell can express polypeptides comprising at least a portion of the anti-PD-1 antibody.

The terms “host cell” or “or a population of host cells” or related terms as used herein refer to a cell (or a population thereof or a plurality of a host cell) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. The host cell (or a population thereof) includes immortalized cell lines. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome or can harbor an extrachromosomal expression vector. In one embodiment, host cells and populations thereof can harbor an extrachromosomal vector that is present after several cell divisions or is present transiently and is lost after several cell divisions.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed or transfected) with a nucleic acid either to be expressed or not to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

Polypeptides of the present disclosure (e.g., antibodies and antigen binding proteins) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, for example from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, E. coli host cells are suitable for expressing small polypeptides. The protein is then purified from culture media or cell extracts. Any of the anti-PD-1 antibodies, or antigen binding protein thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein can be at least 65% pure, at least 75% pure, at least 85% pure, at least 95% pure, or at least 98% pure. Regardless of the exact numerical value of the purity, the polypeptide is sufficiently pure for use as a pharmaceutical product. Any of the anti-PD-1 antibodies, or antigen binding protein thereof, described herein can be expressed by transgenic host cells and then purified to about 65-98% purity or high level of purity using any art-known method.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, a form of glycosylation can be sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

In one embodiment, the antibodies and antigen binding proteins described herein can be modified to become soluble polypeptides which comprises linking the antibodies and antigen binding proteins to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

PEG is a water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a C₁₋₄ alkyl. In one embodiment, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

The serum clearance rate of PEG-modified polypeptide may be modulated (e.g., increased or decreased) by about 10%, 20%, 30%, 40%, 50%, 60%, 70%, 80%, or even 90%, relative to the clearance rate of the unmodified antibodies and antigen binding proteins binding polypeptides. The PEG-modified antibodies and antigen binding proteins may have a half-life (t_{1/2}) which is enhanced relative to the half-life of the unmodified polypeptide. The half-life of PEG-modified polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified antibodies and antigen binding proteins. In some embodiments, the protein half-life is determined in vitro, such as in a buffered saline solution or in serum. In other embodiments, the protein half-life is an in vivo half-life, such as the half-life of the protein in the serum or other bodily fluid of an animal.

The present disclosure provides therapeutic compositions comprising any of the anti-PD-1 antibodies, or antigen binding protein thereof, described herein and a pharmaceutically-acceptable excipient. An excipient encompasses carriers, stabilizers and excipients. Excipients of pharmaceutically acceptable excipients includes for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the anti-PD-1 antibodies (or antigen binding portions thereof) may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding portions thereof) is formulated in the presence of sodium acetate to increase thermal stability.

Any of the anti-PD-1 antibodies (or antigen binding portions thereof) may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the anti-PD-1 antibodies described herein (or antigen binding protein thereof) can be administered to a subject using art-known methods and delivery routes.

The terms “effective amount”, “therapeutically effective amount” or “effective dose” or related terms may be used interchangeably and refer to an amount of antibody or an antigen binding protein (e.g., any of the anti-PD-1 antibodies described herein or antigen binding protein thereof) that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered to a subject at about 0.01 g/kg-50 mg/kg per day, about 0.01 mg/kg-30 mg/kg per day, or about 0.1 mg/kg-20 mg/kg per day. The polypeptide may be administered daily (e.g., once, twice, three times, or four times daily) or less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

The present disclosure provides methods for treating a subject having a disease associated with detrimental expression or over-expression of PD-L1. The disease comprises cancer or tumor cells expressing the tumor-associated antigens. In one embodiment, the cancer or tumor includes cancer of the lung (including non-small cell lung and small cell lung cancers), prostate, breast, ovary, head and neck, thyroid, parathyroid gland, adrenal gland, bladder, intestine, skin, colorectal, anus, rectum, pancreas, leiomyoma, brain, glioma, glioblastoma, esophagus, liver, kidney, stomach, colon, cervix, uterus, fallopian tubes, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the disease or cancer includes Hodgkin's Disease, non-Hodgkin's Disease, chronic or acute leukemias including acute myeloid leukemia, chronic myeloid leukemia, acute lymphoblastic leukemia, chronic lymphocytic leukemia, solid tumors of childhood, lymphocytic lymphoma, Kaposi's sarcoma, and T-cell lymphoma.

The present disclosure provides methods for treating a subject having an inflammatory disorder including intestinal mucosa inflammation wasting diseases associated with colitis, multiple sclerosis, systemic lupus erythematosus, viral infections, rheumatoid arthritis, osteoarthritis, psoriasis, and Crohn's disease.

The present disclosure provides methods for treating a subject having an auto-immune reaction or auto-immune disease, including allergies and asthma.

The present disclosure provides PD-1 binding proteins, particularly anti-PD-1 antibodies, or antigen-binding portions thereof, that specifically bind PD-1 and uses thereof. In one embodiment, the anti-PD-1 antibodies bind an epitope of PD-1 (programmed cell death protein, also known as CD247), for example an extracellular domain of PD-1 protein. In one embodiment, the anti-PD-1 antibodies bind an epitope on PD-1 antigen to block interaction between PD-1 antigen and PD-L1/2 ligands. In one embodiment, the anti-PD-1 antibodies induce release of IFNγ, IL-2, TNFα, IL-4, IL-6 and/or IL-10, for example in an in vitro mixed lymphocyte reaction (MLR) assay. In one embodiment, the anti-PD-1 antibodies specifically bind to PD-1 antigen and exhibit little to no detectable binding to other members of the CD28/CTLA-4 family of T cell regulators including CD28, ICOS, CTLA4 and/or BTLA. In one embodiment, the anti-PD-1 antibodies bind an epitope of PD-1 protein that overlaps with the epitope bound by Keytruda (pembrolizumab) and/or Opdivo (nivolumab).

Various aspects of the anti-PD-1 antibodies relate to antibody fragments, single-chain antibodies, pharmaceutical compositions, nucleic acids, recombinant expression vectors, host cells, and methods for preparing and using such anti-PD-1 antibodies. Methods for using the anti-PD-1 antibodies include in vitro and in vivo methods for binding PD-1 protein, blocking interaction between PD-1 and PD-L1, detecting PD-1, and treating diseases associated with PD-L1 detrimental expression or over-expression.

The present disclosure provides antigen binding proteins that bind specifically to a PD-1 polypeptide (e.g., target antigen) or fragment of the PD-1 polypeptide or PD-1 expressed on a cell. In one embodiment, the antigen binding proteins bind PD-1 expressed on activated T cell, B cells and/or myeloid cells. In one embodiment, the PD-1 target antigen, as a target polypeptide or expressed by a cell, comprises a polypeptide from human (e.g., UniProtKB Q15116; SEQ ID NO:1), cynomolgus monkey (e.g., UniProtKB BOLAJ3; SEQ ID NO:2), rhesus monkey (e.g., UniProtKB BOLAJ2; SEQ ID NO:3), or mouse (e.g., UniProtKB Q02242; SEQ ID NO:4). In one embodiment, the PD-1 target antigen comprises a wild-type or polymorphic or mutant amino acid sequence. The PD-1 target antigen can be prepared by recombinant methods or can be chemically synthesized. The PD-1 target antigen can be in soluble form or membrane-bound form (e.g., expressed by a cell or phage). The PD-1 target antigen can be a fusion protein or conjugated for example with a detectable moiety such as a fluorophore. The PD-1 target antigen can be a fusion protein or conjugated with an affinity tag, such as for example a His-tag.

In one embodiment, wild type and/or mutated human PD-1 antigen can be used in an assay comparing binding capabilities of any of the anti-PD-1 antibodies described herein compared to a control anti-PD-1 antibody, and/or in an epitope mapping assay comparing binding capabilities of any of the anti-PD-1 antibodies described herein compared to a control anti-PD-1 antibody.

The present disclosure provides an anti-PD-1 antibody or antigen-binding fragment which binds an epitope of PD-1 from a human, or can bind (e.g., cross-reactivity) with an epitope of PD-1 (e.g., homologous antigen) from any one or any combination of non-human animals such as mouse, rat, goat, rabbit, hamster, dog and/or monkey (e.g., cynomolgus or rhesus).

In one embodiment, the anti-PD-1 antibody or antigen-binding fragment binds human PD-1 antigen with a binding affinity K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less.

In one embodiment, the anti-PD-1 antibody or antigen-binding fragment binds cynomolgus monkey PD-1 antigen with a binding affinity K_{D} of 10⁻⁵ M or less, or 10⁻⁶ M or less, or 10⁻⁷ M or less, or 10⁻⁸M or less, or 10⁻⁹ M or less, or 10⁻¹⁰ M or less.

In one embodiment, the anti-PD-1 antibody or antigen-binding fragment binds rhesus monkey PD-1 antigen with a binding affinity K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less.

In one embodiment, the anti-PD-1 antibody or antigen-binding fragment binds mouse PD-1 with a binding affinity K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less.

In one embodiment, human PD-1 protein is commercially-available from any one of numerous companies, including for example Sino Biological (e.g., catalog #10377-H08H-B). In one embodiment, cynomolgus PD-1 protein is commercially-available from Sino Biological (e.g., catalog #90311-C08H). In one embodiment, rhesus PD-1 protein is commercially-available from Sino Biological (e.g., catalog #90305-K08H). In one embodiment, mouse PD-1 protein is commercially-available from Sino Biological (e.g., catalog #50124-M08H).

The present disclosure provides a fully human antibody of an IgG class that binds to a PD-1 polypeptide. In one embodiment, the anti-PD-1 antibody comprises a heavy chain variable region having at least 95% sequence identity, or at least 96% sequence identity, or at least 97% sequence identity, or at least 98% sequence identity, or at least 99% sequence identity to the amino acid sequence of SEQ ID NO:5 or 13, or combinations thereof; and/or the anti-PD-1 antibody comprises a light chain variable region having 95% sequence identity, or at least 96% sequence identity, or at least 97% sequence identity, or at least 98% sequence identity, or at least 99% sequence identity to the amino acid sequence of SEQ ID NO:9 or 17, or combinations thereof. In one embodiment, the anti-PD-1 antibody comprises an IgG1, IgG2, IgG3 or IgG4 class antibody. In one embodiment, the anti-PD-1 antibody comprises an IgG1 or IgG4 class antibody.

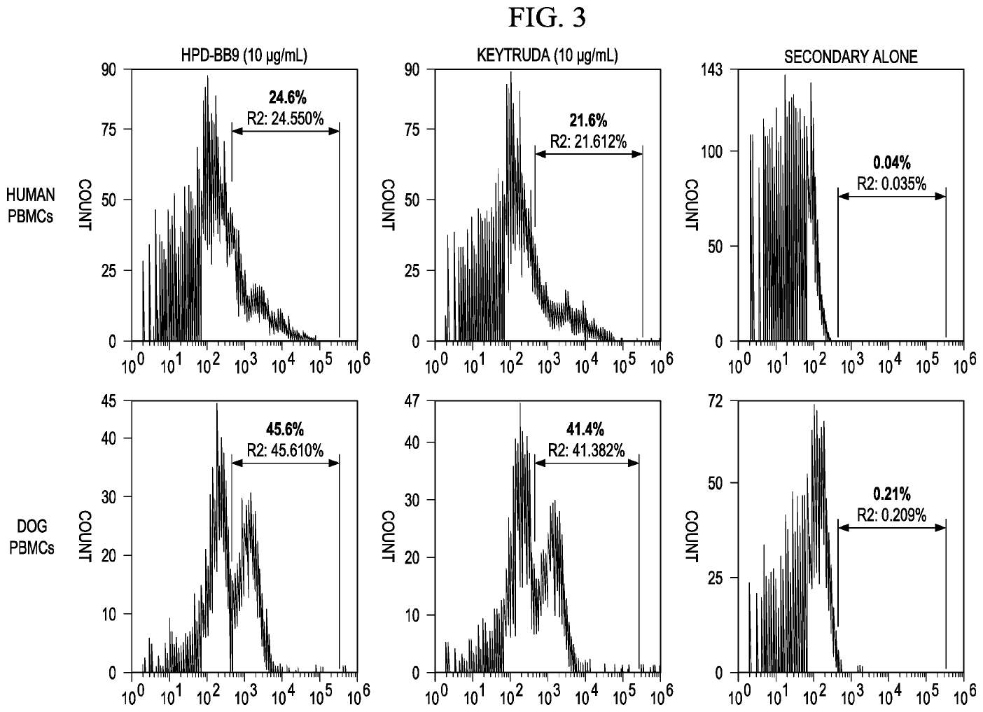

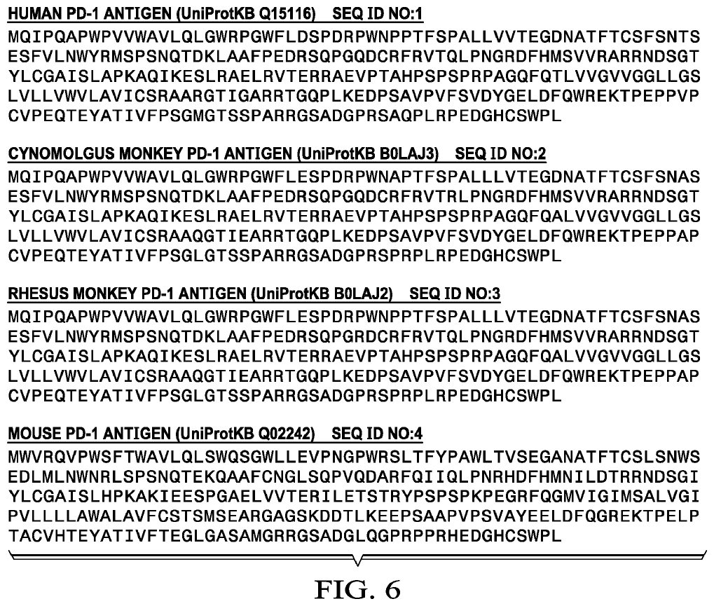

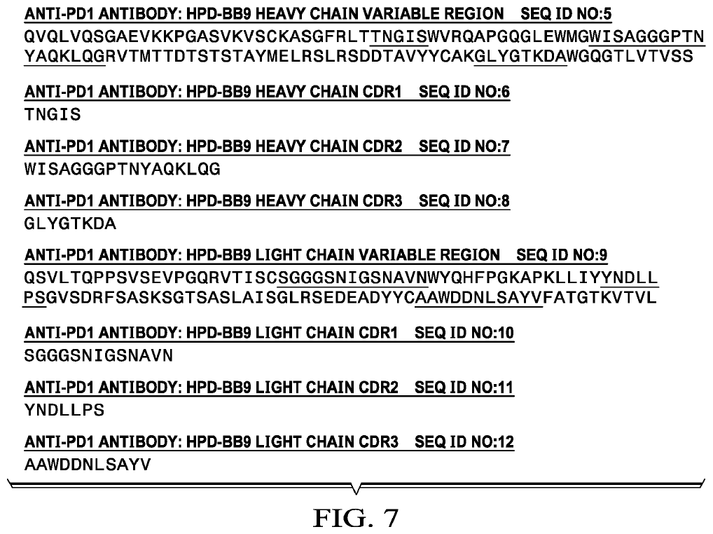

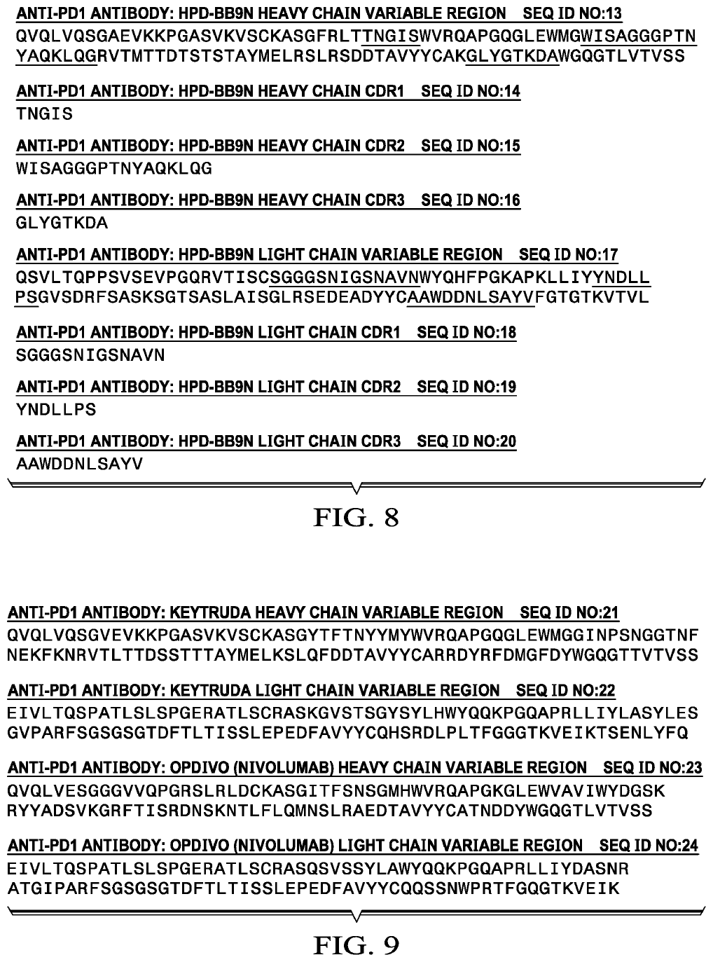

In one embodiment, the anti-PD-1 antibody, or fragment thereof, comprises an antigen binding portion that binds an epitope of a PD-1 target antigen with a binding affinity (K_{D}) of 10⁻⁶ M or less, 10⁻⁷ M or less, 10⁻⁸ M or less, 10⁻⁹ M or less, or 10⁻¹⁰ M or less (see FIGS. 3-10 and Tables 2 and 3). In one embodiment, the PD-1 antigen comprises a cell surface PD-1 antigen or a soluble PD-1 antigen. In one embodiment, the PD-1 antigen comprises an extracellular portion of a cell surface PD-1 antigen. In one embodiment, the PD-1 antigen comprises a human or non-human PD-1 antigen. In one embodiment, the PD-1 antigen is expressed by a human or non-human cell. In one embodiment, the anti-PD-1 antibody binds a human PD-1 expressed by a human PD-1 cell. In one embodiment, binding between the anti-PD-1 antibody, or fragment thereof, can be detected and measured using surface plasmon resonance, flow cytometry and/or ELISA.

The present disclosure provides a fully human antibody comprising both heavy and light chains, wherein the heavy/light chain variable region amino acid sequences have at least 95% sequence identity, or at least 96% sequence identity, or at least 97% sequence identity, or at least 98% sequence identity, or at least 99% sequence identity to any of the following amino acid sequence sets: SEQ ID NOS:5 and 9 (herein called HPD-BB9); or SEQ ID NOS: 13 and 17 (herein called HPD-BB9N).

The present disclosure provides a Fab fully human antibody fragment, comprising a heavy variable region from a heavy chain and a variable region from a light chain, wherein the sequence of the variable region from the heavy chain is at least 95% identical, or at least 96% identical, or at least 97% identical, or at least 98% identical, or at least 99% identical to the amino acid sequence of SEQ ID NO:5 or 13, or combinations thereof. The sequence of the variable region from the light chain is at least 95% identical, or at least 96% identical, or at least 97% identical, or at least 98% identical, or at least 99% identical to the amino acid sequence of SEQ ID NO:9 or 17, or combinations thereof.

The present disclosure provides a Fab fully human antibody fragment, comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS:5 and 9 (herein called HPD-BB9), SEQ ID NOS:13 and 17 (herein called HPD-BB9N).

The present disclosure provides a single chain fully human antibody comprising a polypeptide chain having a variable region from a fully human heavy chain and a variable region from a fully human light chain, and optionally a linker joining the variable heavy and variable light chain regions, wherein the variable heavy region comprises at least 95% sequence identity, or at least 96% sequence identity, or at least 97% sequence identity, or at least 98% sequence identity, or at least 99% sequence identity to the amino acid sequence of SEQ ID NO:5 or 13, or combinations thereof. The variable light region comprises at least 95% sequence identity, or at least 96% sequence identity, or at least 97% sequence identity, or at least 98% sequence identity, or at least 99% sequence identity to the amino acid sequence of SEQ ID NO:9 or 17, or combinations thereof. In one embodiment, the linker comprises a peptide linker having the sequence (GGGGS)_{N} (SEQ ID NO: 31) wherein ‘N’ is 1-6. In one embodiment, the linker comprises a peptide linker having the sequence GGGGSGGGGSGGGGS (SEQ ID NO:25).

The present disclosure provides a single chain fully human antibody comprising a polypeptide chain having heavy chain variable region and a light chain variable region, wherein the heavy/light chain variable region amino acid sequence sets are at least 95% identical, or at least 96% identical, or at least 97% identical, or at least 98% identical, or at least 99% identical to any of the following amino acid sequence sets: SEQ ID NOS:5 and 9 (herein called HPD-BB9), SEQ ID NOS:13 and 17 (herein called HPD-BB9N).

The present disclosure provides pharmaceutical compositions comprising any of the anti-PD-1 antibodies or antigen-binding fragments described herein and a pharmaceutically-acceptable excipient. An excipient encompasses carriers and stabilizers. In one embodiment, the pharmaceutical compositions comprise an anti-PD-1 antibody, or antigen binding fragment thereof, comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS:5 and 9 (herein called HPD-BB9), SEQ ID NOS:13 and 17 (herein called HPD-BB9N).

The present disclosure provides a kit comprising any one or any combination of two or more of the anti-PD-1 antibodies, or antigen binding fragments thereof, described herein. In one embodiment, the kit comprises any one or any combination of two or more anti-PD-1 antibodies, or antigen binding fragments thereof, comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: of SEQ ID NOS:5 and 9 (herein called HPD-BB9); or SEQ ID NOS:13 and 17 (herein call HPD-BB9N). The kit can be used to detect the presence or absence of a PD-1 antigen for example in a biological sample. The kit can be used for conducting an in vitro reaction such as antigen binding assays in the form of ELIZA, flow cytometry or plasmon surface resonance; in vitro cell activation assays including NF-κB activation assays; luciferase-reporter assays; Western blotting and detection; and other such in vitro assays. The kit can be used for treating a subject having a PD1-associated disease or condition, such as multiple myeloma.

The present disclosure provides a first nucleic acid encoding a first polypeptide comprising the anti-PD-1 antibody heavy chain variable region having at least 95% sequence identity, or at least 96% sequence identity, or at least 97% sequence identity, or at least 98% sequence identity, or at least 99% sequence identity with SEQ ID NO:5 or 13.

The present disclosure provides a first nucleic acid encoding a first polypeptide comprising the anti-PD-1 antibody (e.g., HPD-BB9) heavy chain variable region having a heavy chain complementarity determining region 1 (CDR1) having the amino acid sequence of SEQ ID NO:6, a heavy chain CDR2 region having the amino acid sequence of SEQ ID NO:7, and a heavy chain CDR3 region having the amino acid sequence of SEQ ID NO:8.

The present disclosure provides a first nucleic acid encoding a first polypeptide comprising the anti-PD-1 antibody (e.g., HPD-BB9N) heavy chain variable region having a heavy chain complementarity determining region 1 (CDR1) having the amino acid sequence of SEQ ID NO:14, a heavy chain CDR2 region having the amino acid sequence of SEQ ID NO:15, and a heavy chain CDR3 region having the amino acid sequence of SEQ ID NO:16.

The present disclosure provides a first vector operably linked to a first nucleic acid encoding a first polypeptide comprising the anti-PD-1 antibody heavy chain variable region having at least 95% sequence identity, or at least 96% sequence identity, or at least 97% sequence identity, or at least 98% sequence identity, or at least 99% sequence identity with SEQ ID NO:5 or 13. In one embodiment, the first vector comprises an expression vector. In one embodiment, the first vector comprises at least one promoter which is operably linked to the first nucleic acid.

The present disclosure provides a first vector operably linked to a first nucleic acid encoding a first polypeptide comprising the anti-PD-1 antibody (e.g., HPD-BB9) heavy chain variable region having a heavy chain complementarity determining region 1 (CDR1) having the amino acid sequence of SEQ ID NO:6, a heavy chain CDR2 region having the amino acid sequence of SEQ ID NO:7, and a heavy chain CDR3 region having the amino acid sequence of SEQ ID NO:8. In one embodiment, the first vector comprises a first expression vector. In one embodiment, the first vector comprises at least one promoter which is operably linked to the first nucleic acid.

The present disclosure provides a first vector operably linked to a first nucleic acid encoding a first polypeptide comprising the anti-PD-1 antibody (e.g., HPD-BB9N) heavy chain variable region having a heavy chain complementarity determining region 1 (CDR1) having the amino acid sequence of SEQ ID NO:14, a heavy chain CDR2 region having the amino acid sequence of SEQ ID NO:15, and a heavy chain CDR3 region having the amino acid sequence of SEQ ID NO:16. In one embodiment, the first vector comprises a first expression vector. In one embodiment, the first vector comprises at least one promoter which is operably linked to the first nucleic acid.

The present disclosure provides a first host cell harboring the first vector operably linked to the first nucleic acid which encodes the anti-PD-1 antibody heavy chain variable region having at least 95% sequence identity, or at least 96% sequence identity, or at least 97% sequence identity, or at least 98% sequence identity, or at least 99% sequence identity with SEQ ID NO:5 or 13. In one embodiment, the first vector comprises a first expression vector. In one embodiment, the first host cell expresses the first polypeptide comprising the antibody heavy chain variable region having at least 95% sequence identity to the amino acid sequence of SEQ ID NO:5 or 13.

The present disclosure provides a method for preparing a first polypeptide having an antibody heavy chain variable region, the method comprising: culturing a population of the first host cells (e.g., a plurality of the first host cell) harboring the first expression vector under conditions suitable for expressing the first polypeptide having the antibody heavy chain variable region having at least 95% sequence identity to the amino acid sequence of SEQ ID NO:5 or 13. In one embodiment, the method further comprises: recovering from the population of the first host cells the expressed first polypeptide having at least 95% sequence identity to the amino acid sequence of SEQ ID NO:5 or 13.

The present disclosure provides a second nucleic acid encoding a second polypeptide comprising the anti-PD-1 antibody light chain variable region having at least 95% sequence identity, or at least 96% sequence identity, or at least 97% sequence identity, or at least 98% sequence identity, or at least 99% sequence identity with SEQ ID NO:9 or 17.

The present disclosure provides a second nucleic acid encoding a second polypeptide comprising the anti-PD-1 antibody (e.g., HPD-BB9) light chain variable region having a light chain complementarity determining region 1 (CDR1) having the amino acid sequence of SEQ ID NO:10, a light chain CDR2 region having the amino acid sequence of SEQ ID NO:11, and a light chain CDR3 region having the amino acid sequence of SEQ ID NO:12.

The present disclosure provides a second nucleic acid encoding a second polypeptide comprising the anti-PD-1 antibody (e.g., HPD-BB9N) light chain variable region having a light chain complementarity determining region 1 (CDR1) having the amino acid sequence of SEQ ID NO:18, a light chain CDR2 region having the amino acid sequence of SEQ ID NO:19, and a light chain CDR3 region having the amino acid sequence of SEQ ID NO:20.

The present disclosure provides a second vector operably linked to a second nucleic acid encoding a second polypeptide comprising the anti-PD-1 antibody light chain variable region having at least 95% sequence identity, or at least 96% sequence identity, or at least 97% sequence identity, or at least 98% sequence identity, or at least 99% sequence identity with SEQ ID NO:9 or 17. In one embodiment, the second vector comprises a second expression vector. In one embodiment, the second vector comprises at least one promoter which is operably linked to the second nucleic acid.

The present disclosure provides a second vector operably linked to a second nucleic acid encoding a second polypeptide comprising the anti-PD-1 antibody (e.g., HPD-BB9) light chain variable region having a light chain complementarity determining region 1 (CDR1) having the amino acid sequence of SEQ ID NO:10, a light chain CDR2 region having the amino acid sequence of SEQ ID NO:11, and a light chain CDR3 region having the amino acid sequence of SEQ ID NO:12. In one embodiment, the second vector comprises a second expression vector. In one embodiment, the second vector comprises at least one promoter which is operably linked to the second nucleic acid.

The present disclosure provides a second vector operably linked to a second nucleic acid encoding a second polypeptide comprising the anti-PD-1 antibody (e.g., HPD-BB9N) light chain variable region having a light chain complementarity determining region 1 (CDR1) having the amino acid sequence of SEQ ID NO:18, a light chain CDR2 region having the amino acid sequence of SEQ ID NO:19, and a light chain CDR3 region having the amino acid sequence of SEQ ID NO:20. In one embodiment, the second vector comprises a second expression vector. In one embodiment, the second vector comprises at least one promoter which is operably linked to the second nucleic acid.

The present disclosure provides a second host cell harboring the second vector operably linked to the second nucleic acid which encodes the anti-PD-1 antibody light chain variable region having at least 95% sequence identity, or at least 96% sequence identity, or at least 97% sequence identity, or at least 98% sequence identity, or at least 99% sequence identity with SEQ ID NO:9 or 17. In one embodiment, the second vector comprises a second expression vector. In one embodiment, the second host cell expresses the second polypeptide comprising the antibody light chain variable region having at least 95% sequence identity to the amino acid sequence of SEQ ID NO:9 or 17.

The present disclosure provides a method for preparing a second polypeptide having an antibody light chain variable region, the method comprising: culturing a population of the second host cells (e.g., a plurality of the second host cell) harboring the second expression vector under conditions suitable for expressing the second polypeptide having the antibody light chain variable region having at least 95% sequence identity to the amino acid sequence of SEQ ID NO:9 or 17. In one embodiment, the method further comprises: recovering from the population of the second host cells the expressed second polypeptide having at least 95% sequence identity to the amino acid sequence of SEQ ID NO:9 or 17.

The present disclosure provides a first and second nucleic acid, wherein (a) the first nucleic acid encodes a first polypeptide comprising the anti-PD-1 antibody heavy chain variable region having at least 95% sequence identity, or at least 96% sequence identity, or at least 97% sequence identity, or at least 98% sequence identity, or at least 99% sequence identity with SEQ ID NO:5 or 13, and (b) the second polypeptide comprising the anti-PD-1 antibody light chain variable region having at least 95% sequence identity, or at least 96% sequence identity, or at least 97% sequence identity, or at least 98% sequence identity, or at least 99% sequence identity with SEQ ID NO:9 or 17.

The present disclosure provides a vector operably linked to a first and a second nucleic acid, wherein (a) the first nucleic acid encodes a first polypeptide comprising the anti-PD-1 antibody heavy chain variable region having at least 95% sequence identity, or at least 96% sequence identity, or at least 97% sequence identity, or at least 98% sequence identity, or at least 99% sequence identity with SEQ ID NO:5 or 13, and (b) the second polypeptide comprising the anti-PD-1 antibody light chain variable region having at least 95% sequence identity, or at least 96% sequence identity, or at least 97% sequence identity, or at least 98% sequence identity, or at least 99% sequence identity with SEQ ID NO:9 or 17. In one embodiment, the vector comprises an expression vector. In one embodiment, the vector comprises at least a first promoter which is operably linked to the first nucleic acid. In one embodiment, the vector comprises at least a second promoter which is operably linked to the second nucleic acid.

The present disclosure provides a host cell harboring a vector operably linked to a first and second nucleic acid, wherein (a) the first nucleic acid encodes a first polypeptide comprising the anti-PD-1 antibody heavy chain variable region having at least 95% sequence identity, or at least 96% sequence identity, or at least 97% sequence identity, or at least 98% sequence identity, or at least 99% sequence identity with SEQ ID NO:5 or 13, and (b) the second nucleic acid encodes a second polypeptide comprising the anti-PD-1 antibody light chain variable region having at least 95% sequence identity, or at least 96% sequence identity, or at least 97% sequence identity, or at least 98% sequence identity, or at least 99% sequence identity with SEQ ID NO:9 or 17. In one embodiment, the vector comprises an expression vector. In one embodiment, the host cell expresses (a) the first polypeptide comprising the antibody heavy chain variable region having at least 95% sequence identity to the amino acid sequence of SEQ ID NO:5 or 13 and (b) the second polypeptide comprising the antibody light chain variable region having at least 95% sequence identity to the amino acid sequence of SEQ ID NO:9 or 17.

The present disclosure provides a method for preparing a first polypeptide having an antibody heavy chain variable region and a second polypeptide having an antibody light chain variable region, the method comprising: culturing a population of the host cells (e.g., a plurality of the host cell) harboring an expression vector which is operably linked to a first and a second nucleic acid encoding the first and second polypeptides, respectively. In one embodiment, the culturing is conducted under conditions suitable for expressing (a) the first polypeptide having the antibody heavy chain variable region having at least 95% sequence identity to the amino acid sequence of SEQ ID NO:5 or 13, and (b) the second polypeptide having the antibody light chain variable region having at least 95% sequence identity to the amino acid sequence of SEQ ID NO:9 or 17. In one embodiment, the method further comprises: recovering from the population of the host cells the expressed first polypeptide having the antibody heavy chain variable region having at least 95% sequence identity to the amino acid sequence of SEQ ID NO:5 or 13 and the expressed second polypeptide having at least 95% sequence identity to the amino acid sequence of SEQ ID NO:9 or 17.

In one embodiment, the host cell, or population of host cells, harbor one or more expression vectors that can direct transient introduction of the transgene into the host cells or stable insertion of the transgene into the host cells' genome, where the transgene comprises nucleic acids encoding any of the first and/or second polypeptides described herein. The expression vector(s) can direct transcription and/or translation of the transgene in the host cell. The expression vectors can include one or more regulatory sequences, such as inducible and/or constitutive promoters and enhancers. The expression vectors can include ribosomal binding sites and/or polyadenylation sites. In one embodiment, the expression vector, which is operably linked to the nucleic acid encoding the first and/or second polypeptide, can direct production of the first and/or second polypeptide which can be displayed on the surface of the transgenic host cell, or the first and/or second polypeptide can be secreted into the cell culture medium.

The present disclosure provides in vitro and in vivo methods for blocking interaction (e.g., binding) between PD-1 and its cognate ligand PD-L1.

In one embodiment, the methods for blocking interaction between PD-1 polypeptide and PD-L1 polypeptide comprise: contacting any of the anti-PD1 antibodies described herein (e.g., BB9 or BB9N) with a PD-1 polypeptide and a PD-L1 polypeptide, under conditions suitable for binding between the anti-PD1 antibody and the PD-1 polypeptide and for blocking between the PD-1 polypeptide and the PD-L1 polypeptide. In one embodiment, the anti-PD1 antibody can be contacted with the PD-1 polypeptide and the PD-L1 polypeptide at the same time (essentially simultaneously) or sequentially in any order. In one embodiment, the blocking method can be conducted in vitro or in vivo.

In one embodiment, the methods for blocking interaction between a PD-1-expressing cell and a PD-L1-expressing cell comprise: contacting any of the anti-PD1 antibodies described herein (e.g., BB9 or BB9N) with a PD-1-expressing cell and a PD-L1-expressing cell, under conditions suitable for binding between the anti-PD1 antibody and the PD-1-expressing cells and for blocking between the PD-1-expressing cell and the PD-L1-expressing cell. In one embodiment, the anti-PD1 antibody can be contacted with the PD-1-expressing cell and the PD-L1-expressing at the same time (essentially simultaneously) or sequentially in any order. In one embodiment, the blocking method can be conducted in vitro or in vivo. In one embodiment, the PD-1-expressing cell comprises a T cell. In one embodiment, the PD-L1-expressing cell comprises a tumor cell. In one embodiment, the blocking of interaction between the PD-1-expressing cell (e.g., T cell) and the PD-L1-expressing cell (e.g., tumor) by the anti-PD1 antibody blocks activation of a PD-1 receptor on the PD-1-expressing cell. In one embodiment, the blocking of interaction between the PD expressing cell (e.g., T cell) and the PD-L1-expressing cell (e.g., tumor) by the anti-PD1 antibody causes activation of the PD-1-expressing cell (e.g., activation of the T cell).

The present disclosure provides methods for treating a subject having a disease associated with PD-L1 over-expression (or detrimental expression) or a PD-L1-positive cancer, the method comprising: administering to the subject an effective amount of a therapeutic composition comprising an anti-PD-1 antibody described herein or antigen binding fragment thereof, e.g., which is selected from a group consisting of any of the fully human anti-PD-1 antibodies described herein, any of the Fab fully human anti-PD-1 antibodies described herein, and any of the single chain human anti-PD-1 antibodies described herein.

In one embodiment, the disease or cancer associated with PD-L1 over-expression (or detrimental expression) comprises: cancer of the lung (including non-small cell lung and small cell lung cancers), prostate, breast, ovary, head and neck, thyroid, parathyroid gland, adrenal gland, bladder, intestine, skin, colorectal, anus, rectum, pancreas, leiomyoma, brain, glioma, glioblastoma, esophagus, liver, kidney, stomach, colon, cervix, uterus, fallopian tubes, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the disease or cancer includes Hodgkin's Disease, non-Hodgkin's Disease, chronic or acute leukemias including acute myeloid leukemia, chronic myeloid leukemia, acute lymphoblastic leukemia, chronic lymphocytic leukemia, solid tumors of childhood, lymphocytic lymphoma, Kaposi's sarcoma, and T-cell lymphoma.

The present disclosure provides methods for treating a subject having an inflammatory disorder including intestinal mucosa inflammation wasting diseases associated with colitis, multiple sclerosis, systemic lupus erythematosus, viral infections, rheumatoid arthritis, osteoarthritis, psoriasis, and Crohn's disease.

The present disclosure provides methods for treating a subject having an auto-immune reaction or auto-immune disease, including allergies and asthma.

### EXAMPLES

The following examples are meant to be illustrative and can be used to further understand embodiments of the present disclosure and should not be construed as limiting the scope of the present teachings in any way.

### Example 1: Measuring Binding Affinities Using Surface Plasmon Resonance

Binding kinetics of anti-PD1 antibodies with PD-1 proteins from different species was measured using surface plasmon resonance (SPR). The anti-PD1 antibodies tested included HPD-BB9, and commercially-obtained anti-PD1 antibodies Keytruda and Opdivo. The PD-1 proteins from human (catalog #10377-H08H-B), cynomolgus monkey (catalog #90311-C08H), rhesus monkey (catalog #90305-K08H), and mouse (catalog #50124-M08H) were obtained from Sino Biological. Anti-human fragment crystallizable region (Fc region) antibody was immobilized on a CMS sensor chip to approximately 8,000 RU using standard N-hydroxysuccinimide/N-Ethyl-N′-(3-dimethylaminopropyl) carbodiimide hydrochloride (NHS/EDC) coupling methodology. The anti-PD1 antibody HPD-BB9 (2 μg/mL) were captured for 60 seconds at a flow rate of 10 μL/minute. Recombinant human, cyno, rhesus and mouse PD-1-His were serially diluted in a running buffer of 0.01 M HEPES pH 7.4, 0.15 M NaCl, 3 mM EDTA, 0.05% v/v Surfactant P20 (HBS-EP+). All measurements were conducted in HBS-EP+ buffer with a flow rate of 30 μL/minute. A 1:1 (Langmuir) binding model was used to fit the data. All BIACORE assays were performed at room temperature.

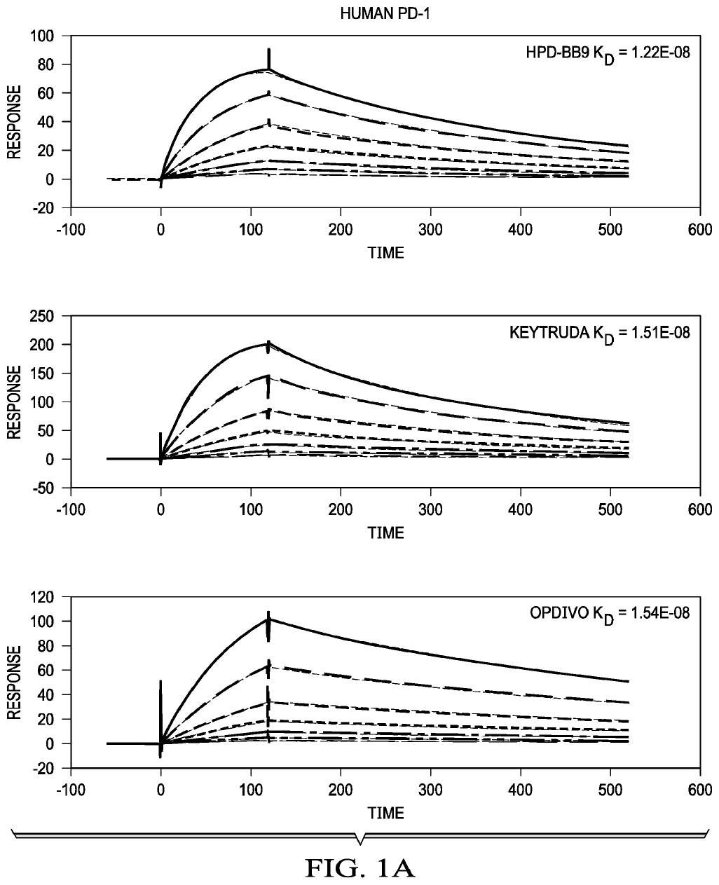

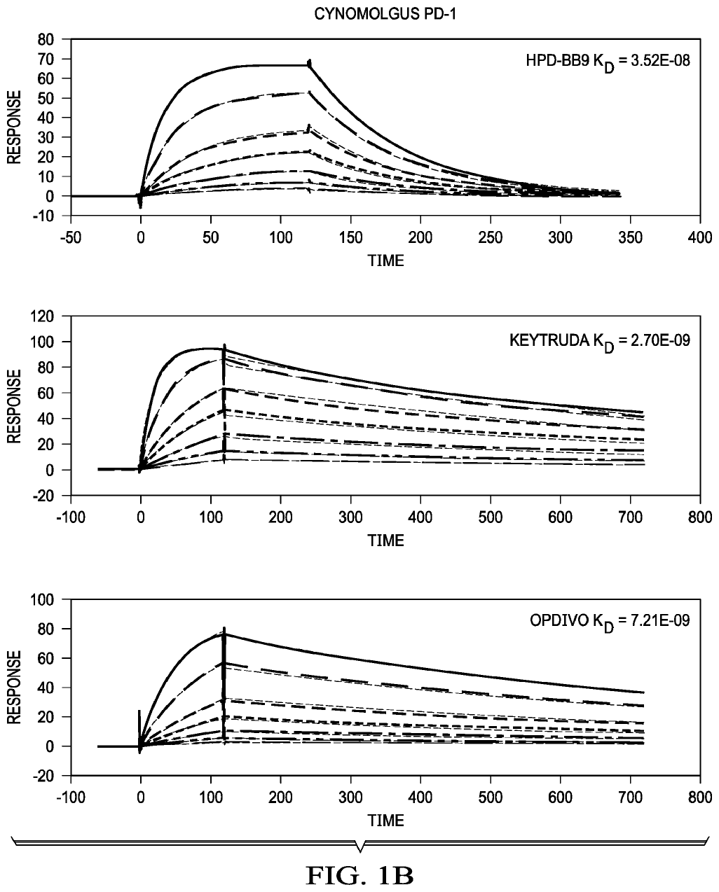

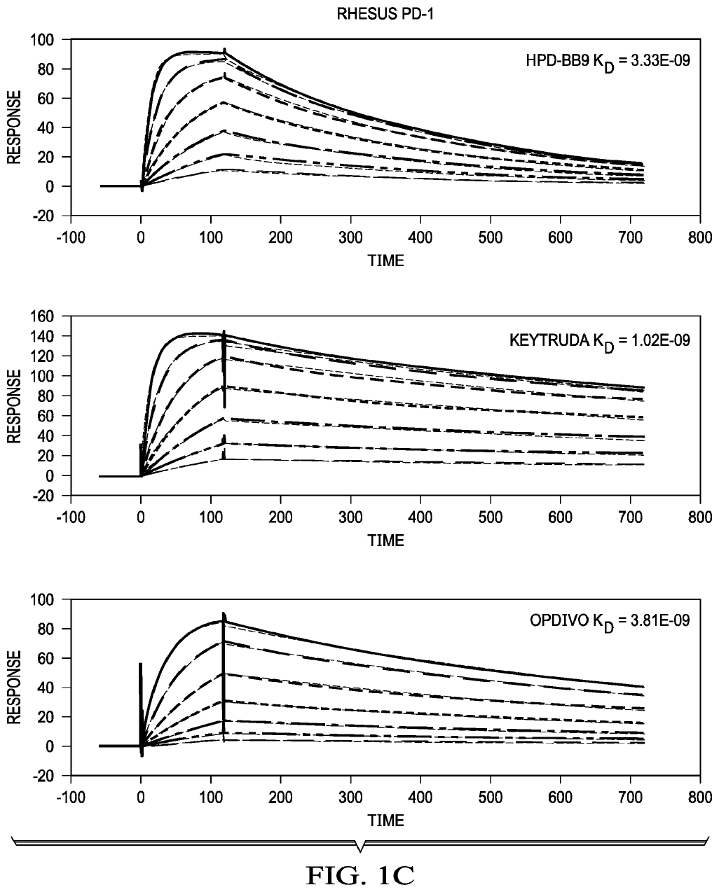

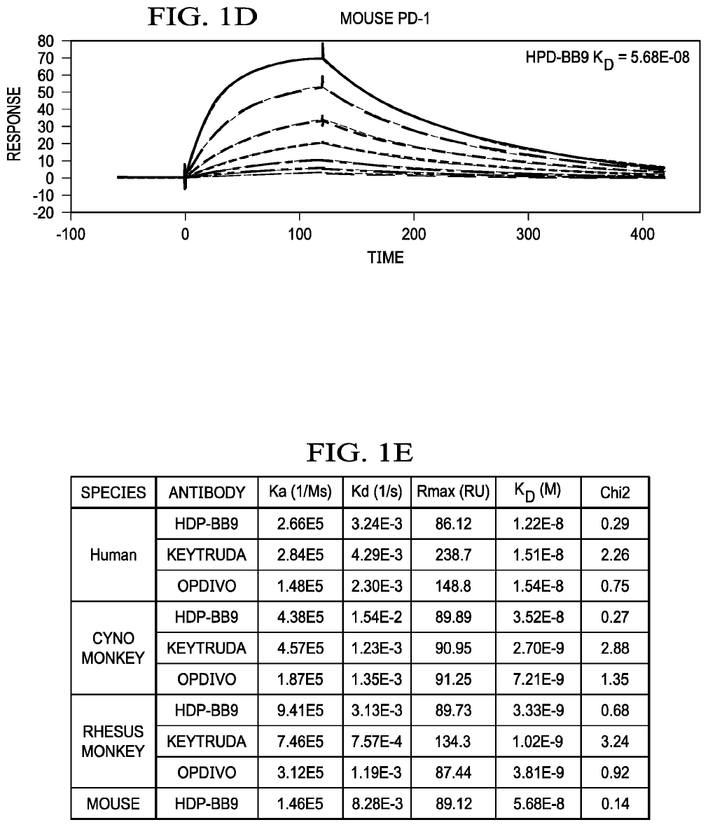

The SPR sensorgrams of anti-PD1 antibodies HPD-BB9, Keytruda and Opdivo are shown in FIGS. 1A-D, and their corresponding binding kinetics are listed in the table shown in FIG. 1E.

### Example 2: Cell Binding Assay by Flow Cytometry

The human PD-1 (hPD-1)-expressing RAJI cell line (Invivogen; Cat. code: raji-hpd1; lot. 40-01-rajihpd1) was cultured in complete IMDM (IMDM, 10% heat-inactivated FCS, Pen/Strep) supplemented with 10 μg/mL Blasticidin. Wild-type (WT) RAJI cells were cultured in complete IMDM.

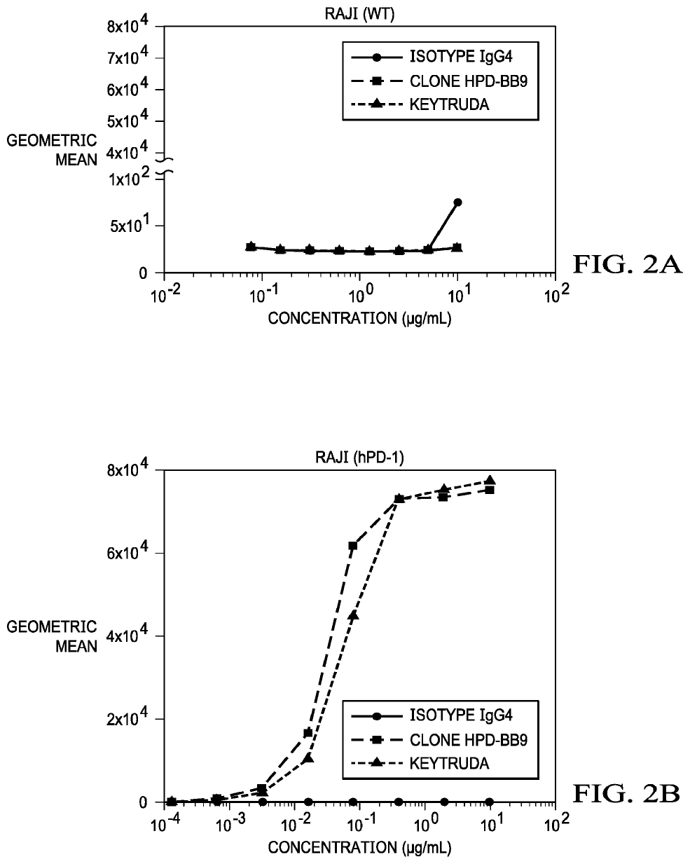

Cells were plated at 80,000 cells/well in a V-bottom 96-well plate and washed twice using 170 μL/well of FACS buffer (PBS 1×, 2% heat-inactivated FCS, 0.1% sodium azide). Anti-PD-1 (clone HPD-BB9 or competitor Keytruda) and isotype control antibodies were diluted in FACS buffer at various concentrations (ranging from 10 to 0.000128 μg/mL) and incubated with either WT or hPD-1-expressing Jurkat cells in 100 μL/well for 30 min at 4° C. After 2 washes in 150 μL/well of FACS buffer, cells were incubated with 70 μL/well of an APC-conjugated anti-human Fc-specific IgG secondary antibody (Biolegend; Cat. no. 409306, lot. B232398; dilution 1:17.5 in FACS buffer) for 20 min at 4° C. Cells were washed twice, resuspended in 120 μL/well of FACS buffer and acquired by flow cytometry on the Attune NxT. Data were analyzed by using FlowJo v10. The Raji (WT) results are shown in FIG. 2A, and the Raji (hPD-1) result are shown in FIG. 2B.

### Example 3: Cell Binding on Human and Canine PBMCs

Human or canine peripheral blood mononuclear cells (PBMCs) were plated at 100,000 cells/well in a V-bottom 96-well plate and washed twice using 170 μL/well of FACS buffer (PBS 1×, 2% heat-inactivated FCS, 2 mM EDTA).

Anti-PD-1 clone HPD-BB9 or Keytruda antibodies were diluted in FACS buffer at 10 μg/mL and incubated with either human or dog PBMCs in 50 μL/well for 30 min at 4° C. After 2 washes in 170 μL/well of FACS buffer, cells were incubated with 50 μL/well of an AF647-conjugated mouse anti-human IgG Fc-specific secondary antibody (Biolegend; Cat. no. 409320; dilution 1:200 in FACS buffer) for 20 min at 4° C. Some PBMCs were only stained with the secondary antibody alone (secondary alone) at the same dilution (1:200) as negative control. Cells were washed twice, fixed in 100 μL of fixation buffer for 20 minutes at room temperature in the dark. Then, cells were washed once and resuspended in 200 μL/well of FACS buffer and acquired by flow cytometry on the Attune NxT. Data were analyzed by using FlowJo v10. The flow cytometry results are shown in FIG. 3.

### Example 4: Mixed Lymphocyte Reaction (MLR) Assay

CD14⁺ cells were isolated from fresh human peripheral blood mononuclear cells (PBMCs) using anti-CD14 biotin antibody (Biolegend; Cat No. 325624) in conjunction with anti-biotin beads (Miltenyi; Cat No. 130-042-401) and a LS separation column (Miltenyi; Cat No. 130-042-401). The CD14⁺ cells were resuspended in complete IMDM media supplemented with 10% FBS, GM-CSF (Biolegend; Cat No. 572903 at 150 ng/mL) and IL-4 (Biolegend; Cat No. 574004 at 150 ng/mL). Cells were then plated out at 2.0E+06 cells/well in 4 mL of media in a 12-well plate and placed in a 37° C. incubator for 7 days. On day 7, total T cells were isolated from human PBMCs of a different donor using a Miltenyi PAN T-Cell isolation Kit, human (Miltenyi; Cat No. 130-096-535) and mixed with the pre-cultured CD14⁺-derived dendritic cells at a ratio of 5.0E+05 T cells/well to 5.0E+04 dendritic cells/well. This mixture was plated out in 100 μL/well of IMDM media supplemented with 10% FBS. In addition, 100 μL of a 2× concentration of the antibodies was added to their respective wells. The tested antibodies included HPD-BB9, Keytruda, Opdivo and control human IgG4 isotype. The plate was placed back in the 37° C. incubator for 5 days.

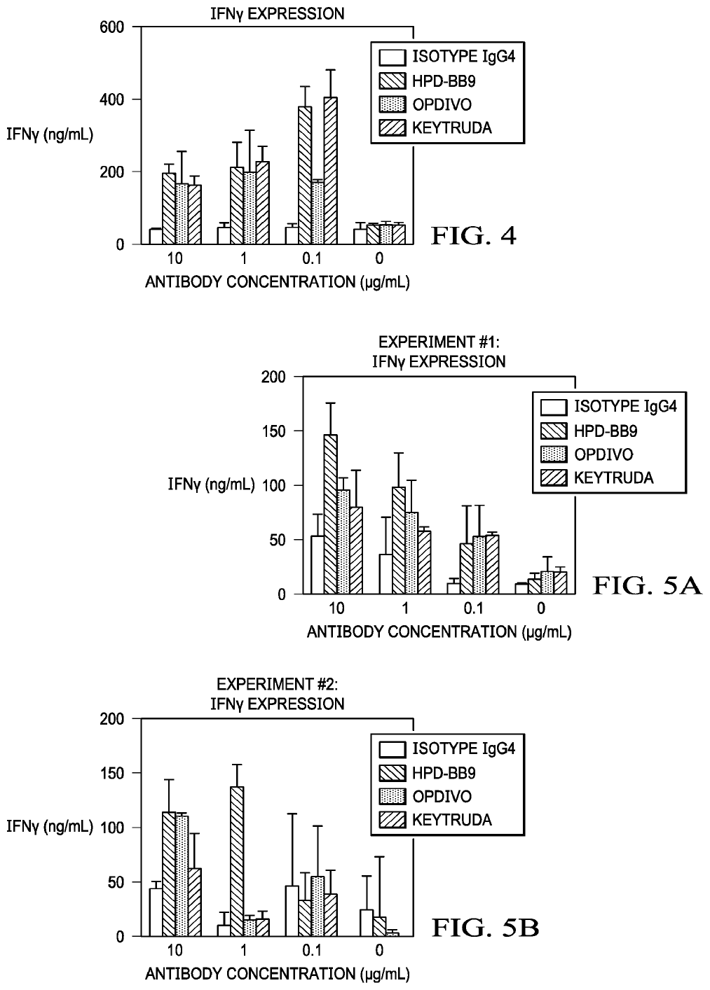

On day 5 post-co-culture, the cells were spun at 300 g for 5 minutes and the supernatants were collected and the IFNγ content of each well measured using the proinflammatory panel 1 (human) kit from Meso Scale Discovery (MSD; Cat. No. K15049D) by following the manufacturer recommendations. The results of interferon gamma release is shown in FIG. 4.

### Example 5: Three-Way Mixed Lymphocyte Reaction (MLR) Assay

On day 0, peripheral blood mononuclear cells (PBMCs) from three different human healthy donors were prepared and resuspended into complete RPMI-10AB medium (RPMI1640 supplemented with 10% human AB serum from Life Technologies; Cat. No. 340055). An equal number of PBMCs from each donor was plated on a flat-bottom 96-well plate to obtain a 1.65E+05 cells/donor/well (˜5.0E+05 cells/well final) seeding density in 200 μL of RPMI-10AB. Isotype control or anti-PD-1 (clone HPD-BB9 or Keytruda) human IgG₄ antibodies were diluted in complete RPMI-10AB medium at a 2× concentration (20, 2 or 0.2 μg/mL), then subsequently 100 μL/well was added to the appropriate wells for a final concentration of 10, 1 or 0.1 μg/mL. The plate was incubated for 5 days in a humidified tissue culture incubator (37° C., 5% CO₂).

On day 5 post-co-culture, the cells were spun at 300 g for 5 minutes and the supernatants were collected and the IFNγ content of each well measured using the proinflammatory panel 1 (human) kit from Meso Scale Discovery (MSD; Cat. No. K15049D) by following the manufacturer recommendations.

Data are shown as IFNγ concentrations from two independent experiments performed with 6 different blood healthy donors (3 for each experiment). The results of the first and second experiments are shown in FIGS. 5A and B, respectively.

### Example 6: PD1/PD-L1 Blockade Reporter Bioassay

Functional activity of anti-PD1 antibody clones HPD-BB9, KEYTRUDA and isotype control IgG4 antibody from Biolegend (Cat. No. 403702) was evaluated using the PD1/PD-L1 blockade assay from Promega (Cat. No. J1250) as recommended by the manufacturer. The antibodies were diluted in assay buffer (RPMI1640+1% FBS) incubated at concentrations ranging from 10 to 0.0024 μg/mL (1:4 serial dilutions).

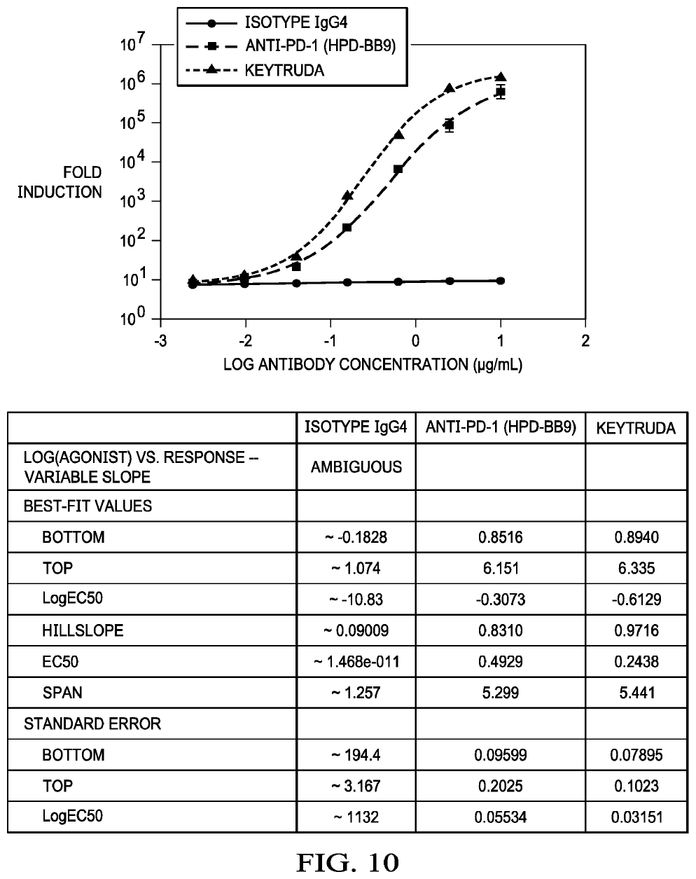

FIG. 10 shows dose-dependent increase in luciferase signal (RLU, relative light unit) detected when the anti-PD1 antibodies blocked the interaction between PD1 and PD-L1.

Data represents an average of duplicate values and are given as a fold induction of Relative Light Units (RLU) which was calculated as follows: [RLU_{(induced-background)}/RLU_{(no antibody control-background)}].

Human anti-hPD-1 clone HPD-BB9 shows a functional dose-dependent blockade of PD-1/PD-L1 interaction with an EC50 of 0.4929 μg/mL similar to KEYTRUDA EC50 of 0.2438 μg/mL.

### Example 7: In Vivo Efficacy Study of Anti-PD1 Clone HPD-BB9 in MB-49 Syngeneic Tumor Model

Anti-tumor activity of human anti-PD1 clone HPD-BB9 was evaluated in MB-49 syngeneic tumor model. C57BL/6 mice were inoculated subcutaneously into the right flank with 1.0E+05 MB-49 mouse bladder tumor cells prepared in HBSS 1× (100 μL/mouse) and randomized into three different treatment groups on day 7 (when a tumor bump was present in more than 80% of the animals). If a mouse did not present a tumor bump at treatment start, it was removed from the study.

Anti-PD-1 human IgG4 clone HPD-BB9 (n=10 mice) at 5 or 15 mg/kg and isotype human IgG4 control (n=10 mice) at 15 mg/kg were administered systemically by subcutaneous injections (150 μL/mouse) on day 7, 10 and 13 post tumor inoculation.

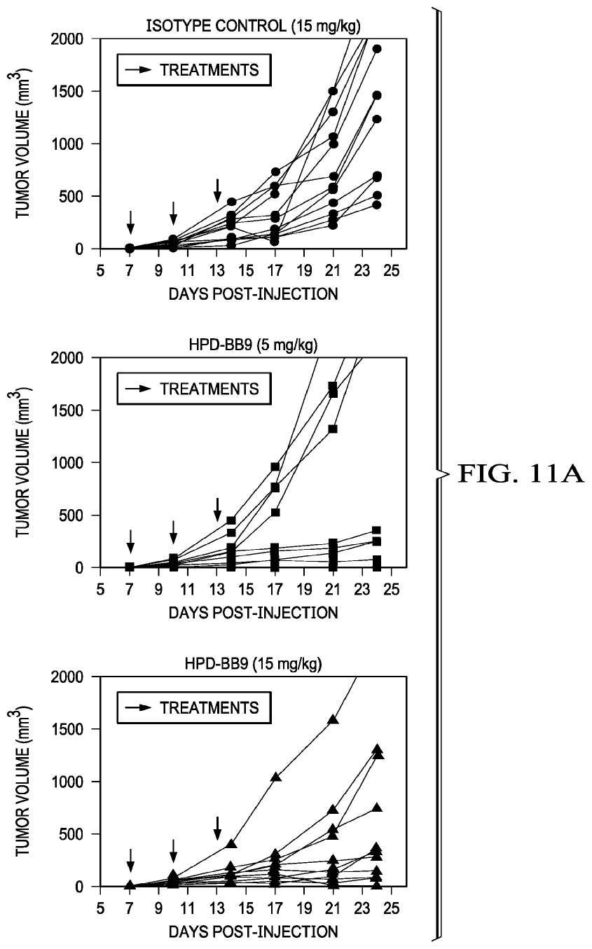

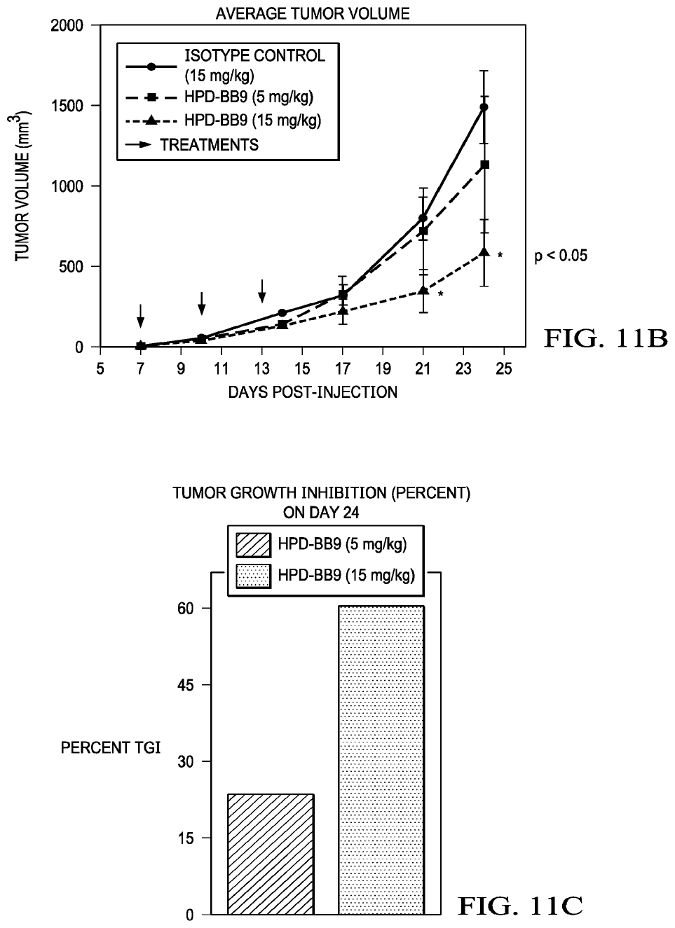

FIG. 11A shows the effect of 5 and 15 mg/kg of HPD-BB9 clone and 15 mg/kg of isotype control on the tumor volume of each individual mouse, measured over 24 days. FIG. 11B shows the effect of 5 and 15 mg/kg of HPD-BB9 clone and 15 mg/kg of isotype control on the tumor volume—averaged for the 10 mice, measured over 24 days. FIG. 11C shows percent tumor growth inhibition by HPD-BB9 clone (TGI=(1−[mean HPD-BB9/mean Isotype])×100) calculated at the end of the study day 24 post tumor cell implantation. Human Anti-PD-1 Clone HPD-BB9 shows anti-tumor activity at 15 mg/kg in the MB-49 syngeneic tumor model.

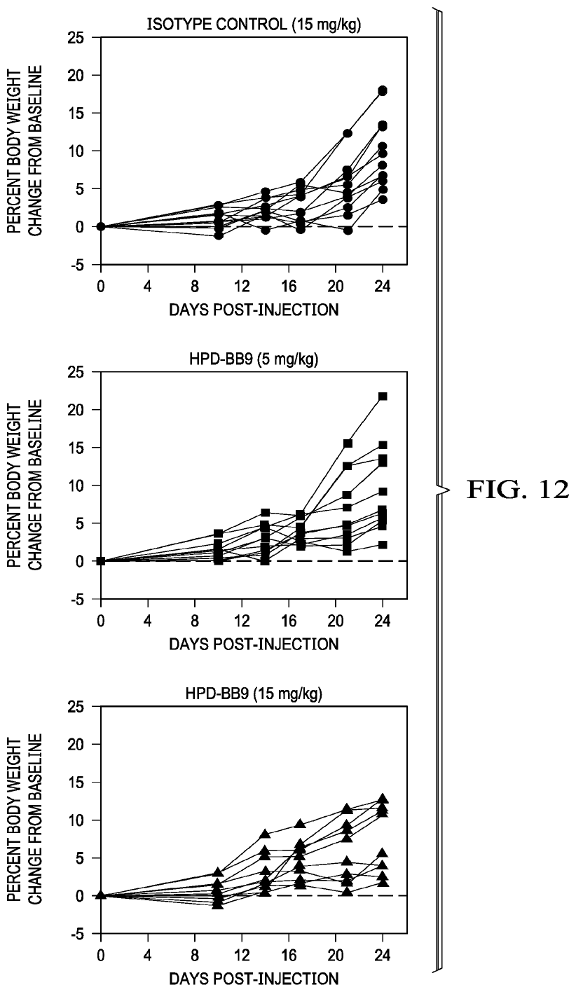

FIG. 12 shows the percent body weight change from baseline (day 0). The body weight of each mouse was collected on day 0 and at several time points during the course of the study, then a percent body weight change from baseline (day 0) was calculated. *p<0.05 is considered statistically significant.

What is claimed:

1. A fully human anti-PD-1 antibody, or an antigen-binding fragment thereof, comprising a heavy chain and a light chain, the heavy chain and the light chain comprising:
a) a heavy chain complementarity determining region 1 (CDR1) having the amino acid sequence of SEQ ID NO:6, a heavy chain CDR2 having the amino acid sequence of SEQ ID NO:7, a heavy chain CDR3 having the amino acid sequence of SEQ ID NO:8, a light chain CDR1 having the amino acid sequence of SEQ ID NO:10, a light chain CDR2 having the amino acid sequence of SEQ ID NO:11, and a light chain CDR3 having the amino acid sequence of SEQ ID NO:12 (e.g., herein called HPD-BB9); or
b) a heavy chain complementarity determining region 1 (CDR1) having the amino acid sequence of SEQ ID NO:14, a heavy chain CDR2 having the amino acid sequence of SEQ ID NO:15, a heavy chain CDR3 having the amino acid sequence of SEQ ID NO:16, a light chain CDR1 having the amino acid sequence of SEQ ID NO:18, a light chain CDR2 having the amino acid sequence of SEQ ID NO:19, and a light chain CDR3 having the amino acid sequence of SEQ ID NO:20 (e.g., herein called HPD-BB9N).

2. A fully human anti-PD-1 antibody, or an antigen-binding fragment thereof, comprising;
a) a heavy chain and a light chain, the heavy chain comprising a heavy chain variable region having at least 95% sequence identity to the amino acid sequence of SEQ ID NO:5; and the light chain comprising a light chain variable region having at least 95% sequence identity to the amino acid sequence of SEQ ID NO:9; or
b) a heavy chain and a light chain, the heavy chain comprising a heavy chain variable region having at least 95% sequence identity to the amino acid sequence of SEQ ID NO:13; and the light chain comprising a light chain variable region having at least 95% sequence identity to the amino acid sequence of SEQ ID NO:17.

3. The fully human anti-PD-1 antibody, or the antigen-binding fragment thereof, of claim 2, wherein
a) the heavy chain variable region and the light chain variable region comprise the amino acid sequences of SEQ ID NOS:5 and 9 (e.g., herein called HPD-BB9); or
b) wherein the heavy chain variable region and the light chain variable region comprise the amino acid sequences of SEQ ID NOS:13 and 17 (e.g., herein called HPD-BB9N).

4. The fully human anti-PD-1 antibody, or the antigen-binding fragment thereof, of claim 2, wherein the antigen-binding fragment is a Fab fragment comprising a variable domain region from a heavy chain and a variable domain region from a light chain, wherein
a) the variable domain region from the heavy chain comprises a sequence having at least 95% sequence identity to the amino acid sequence of SEQ ID NO:5, and wherein the variable domain region from the light chain comprises a sequence having at least 95% sequence identity to the amino acid sequence of SEQ ID NO:9; or
b) the variable domain region from the heavy chain comprises a sequence having at least 95% sequence identity to the amino acid sequence of SEQ ID NO:13, and wherein the variable domain region from the light chain comprises a sequence having at least 95% sequence identity to the amino acid sequence of SEQ ID NO:17.

5. The Fab fragment of claim 4, wherein
a) the variable domain region from the heavy chain and the variable domain region from the light chain are SEQ ID NOS:5 and 9 (e.g., herein called HPD-BB9); or
b) the variable domain region from the heavy chain and the variable domain region from the light chain are SEQ ID NOS:13 and 17 (e.g., herein called HPD-BB9N).

6. The fully human anti-PD-1 antibody, or the antigen-binding fragment thereof, of claim 2, wherein the antigen-binding fragment is a single chain antibody comprising variable domain region from a heavy chain and a variable domain region from a light chain joined together with a peptide linker, wherein
a) the variable domain region from the heavy chain comprises a sequence having at least 95% sequence identity to the amino acid sequence of SEQ ID NO:5, and wherein the variable domain region from the light chain comprises a sequence having at least 95% sequence identity to the amino acid sequence of SEQ ID NO:9; or
b) the variable domain region from the heavy chain comprises a sequence having at least 95% sequence identity to the amino acid sequence of SEQ ID NO:13, and wherein the variable domain region from the light chain comprises a sequence having at least 95% sequence identity to the amino acid sequence of SEQ ID NO:17.

7. The single chain human anti-PD-1 antibody of claim 6, wherein
a) the variable domain region from the heavy chain and the variable domain region from the light chain are SEQ ID NOS:5 and 9 (e.g., herein called HPD-BB9); or
b) the variable domain region from the heavy chain and the variable domain region from the light chain are SEQ ID NOS:13 and 17 (e.g., herein called HPD-BB9N).

8. The fully human anti-PD-1 antibody, or the antigen-binding fragment thereof, of any of the preceding claims, comprising an IgG1, IgG2, IgG3 or IgG4 antibody.

9. The fully human anti-PD-1 antibody, or the antigen-binding fragment thereof, of any of the preceding claims, comprising an IgG1 or IgG4 isotype antibody.

10. The fully human anti-PD-1 antibody, or the antigen-binding fragment thereof, of any of the preceding claims that blocks binding of PD-1 protein to human PD-L1 protein.

11. The fully human anti-PD-1 antibody, or the antigen-binding fragment thereof, of any of the preceding claims which binds to human PD-1 protein and cross-reacts with PD-1 protein from any one or any combination of cynomolgus monkey, rhesus monkey, mouse and/or dog.

12. The fully human anti-PD-1 antibody, or the antigen-binding fragment thereof, of any of the preceding claims which binds to human PD-1 protein and does not cross-react with PD-1 protein from any one or any combination of cynomolgus monkey, rhesus monkey, mouse and/or dog.

13. The fully human anti-PD-1 antibody, or the antigen-binding fragment thereof, of any of the preceding claims which binds to human PD-1 protein expressed on the surface of human cells.

14. The fully human anti-PD-1 antibody, or the antigen-binding fragment thereof, of any of the preceding claims which binds human PD-1 protein with a K_{D} of 10⁻⁷ M or less.

15. The human anti-PD-1 antibody, or the antigen-binding fragment thereof, of any of the preceding claims which binds cynomolgus monkey PD-1 protein with a K_{D} of 10⁻⁷ M or less.

16. The human anti-PD-1 antibody, or the antigen-binding fragment thereof, of any of the preceding claims which binds rhesus monkey PD-1 protein with a K_{D} of 10⁻⁸ M or less.

17. The human anti-PD-1 antibody, or the antigen-binding fragment thereof, of any of the preceding claims which binds mouse PD-1 protein with a K_{D} of 10⁻⁷ M or less.

18. A pharmaceutical composition, comprising a pharmaceutically-acceptable excipient and the human anti-PD-1 antibody or antigen-binding fragment of any one of the preceding claims.

19. A kit comprising the human anti-PD-1 antibody of any one of claims 1-13.

20. A first nucleic acid that encodes a first polypeptide having the heavy chain variable region of the human anti-PD-1 antibody of any one of claims 1-7.

21. A second nucleic acid that encodes a second polypeptide having the light chain variable region of the human anti-PD-1 antibody of any one of claims 1-7.

22. A first nucleic acid that encodes a first polypeptide having the heavy chain variable region of the human anti-PD-1 antibody of any one of claims 1-7, and a second nucleic acid that encodes a second polypeptide having the light chain variable region of the human anti-PD1 antibody of any one of claims 1-7.

23. A nucleic acid that encodes a single chain antibody comprising a polypeptide having the heavy chain variable region of the human anti-PD-1 antibody of any one of claims 1-7, and encodes the light chain variable region of the human anti-PD-1 antibody of any one of claims 1-7.

24. A first vector comprising the first nucleic acid of claim 20.

25. A second vector comprising the second nucleic acid of claim 21.

26. A (single) vector comprising the first and second nucleic acids of claim 22.

27. A first vector comprising the first nucleic acid of claim 22 and a second vector comprising the second nucleic acid of claim 22.

28. A vector comprising the nucleic acid of claim 23.

29. A first host cell harboring the first vector of claim 24.

30. The first host cell of claim 29, wherein the first vector comprises a first expression vector, and wherein the first host cell expresses the first polypeptide comprising the heavy chain variable region.

31. A second host cell harboring the second vector of claim 25.

32. The second host cell of claim 31, wherein the second vector comprises a second expression vector, and wherein the second host cell expresses the second polypeptide comprising the light chain variable region.

33. A host cell harboring the (single) vector of claim 26.

34. The host cell of claim 33, wherein the (single) vector comprises an expression vector, wherein the host cell expresses the first polypeptide comprising the heavy chain variable region and expresses the second polypeptide comprising the light variable region.

35. A host cell harboring the first vector of claim 27 and harboring the second vector of claim 27.

36. The host cell of claim 35, wherein the first vector comprises a first expression vector and the second vector comprises a second expression vector, and wherein the host cell expresses the first polypeptide comprising the heavy chain variable region and expresses the second polypeptide comprising the light variable region.

37. A host cell harboring the (single) vector of claim 28.

38. The host cell of claim 37, wherein the (single) vector comprises an expression vector, and wherein the host cell expresses the single chain antibody comprising a polypeptide having the heavy chain variable region and the light variable region.

39. A method for preparing a first polypeptide having an antibody heavy chain variable region, the method comprising: culturing a population of the host cell of claim 30 under conditions suitable for expressing the first polypeptide having the antibody heavy chain variable region.

40. The method of claim 39, further comprising: recovering from the host cells the expressed first polypeptide having the antibody heavy chain variable region.

41. A method for preparing a polypeptide having an antibody light chain variable region, the method comprising: culturing a population of the host cell of claim 32 under conditions suitable for expressing the second polypeptide having the antibody light chain variable region.

42. The method of claim 41, further comprising: recovering from the host cells the expressed second polypeptide having the antibody light chain variable region.

43. A method for preparing a first polypeptide having the antibody heavy chain variable region and a second polypeptide having the antibody light chain variable region, the method comprising: culturing a population of the host cell of claim 34 under conditions suitable for expressing the first polypeptide having the antibody heavy chain variable region and the second polypeptide having the antibody light chain variable region.

44. The method of claim 43, further comprising: recovering from the host cells the expressed first polypeptide having the antibody heavy chain variable region and the expressed second polypeptide having the antibody light chain variable region.

45. A method for preparing a first polypeptide having an antibody heavy chain variable region and a second polypeptide having an antibody light chain variable region, the method comprising: culturing a population of the host cell of claim 36 under conditions suitable for expressing the first polypeptide having the antibody heavy chain variable region and a second polypeptide having the antibody light chain variable region.

46. The method of claim 45, further comprising: recovering from the host cells the expressed first polypeptide having the antibody heavy chain variable region and the expressed second polypeptide having the antibody light chain variable region.

47. A method for preparing a single chain antibody having a heavy chain variable region and a light chain variable region, the method comprising: culturing a population of the host cell of claim 38 under conditions suitable for expressing polypeptide comprising the heavy chain variable region and the light chain variable region.

48. The method of claim 47, further comprising: recovering from the host cells the expressed polypeptide comprising the heavy chain variable region and the light chain variable region.

49. A method (e.g., in vitro or in vivo method) for blocking interaction between PD-1 polypeptide and PD-L1 polypeptide comprising: contacting any one of the anti-PD1 antibodies of claims 1-7 with a PD-1 polypeptide and a PD-L1 polypeptide, under conditions suitable for binding between the anti-PD1 antibody and the PD-1 polypeptide and for blocking between the PD-1 polypeptide and the PD-L1 polypeptide.

50. A method (e.g., in vitro or in vivo method) for blocking interaction between a PD-1-expressing cell and a PD-L1-expressing cell comprising: contacting any of the anti-PD1 antibodies of claims 1-7 with a PD-1-expressing cell and a PD-L1-expressing cell, under conditions suitable for binding between the anti-PD1 antibody and the PD-1-expressing cells and for blocking between the PD-1-expressing cell and the PD-L1-expressing cell.

51. The method of claim 50, wherein the PD-1-expressing cell comprises a T cell.

52. The method of claim 50, wherein the PD-L1-expressing cell comprises a tumor cell.

53. The method of claim 50, wherein the blocking of the interaction between the PD-1-expressing cell (e.g., T cell) and the PD-L1-expressing cell (e.g., tumor) by the anti-PD1 antibody blocks activation of a PD-1 receptor on the PD-1-expressing cell.

54. The method of claim 50, wherein the blocking of the interaction between the PD-1-expressing cell (e.g., T cell) and the PD-L1-expressing cell (e.g., tumor) by the anti-PD1 antibody causes activation of the PD-1-expressing cell (e.g., activation of the T cell).

55. A method for treating a subject having a disease associated with PD-L1 over-expression or PD-L1 detrimental expression, the method comprising: administering to the subject an effective amount of a therapeutic composition comprising the human anti-PD-1 antibody of any one of claims 1-7.

56. The human anti-PD-1 antibody of any one of claims 1-7, for use in treating a disease associated with PD-L1 over-expression or PD-1 detrimental expression.

57. The method of claim 55, wherein the disease associated with PD-L1 over-expression or PD-L1 detrimental expression is selected from a group consisting of cancer of the lung (including non-small cell lung and small cell lung cancers), prostate, breast, ovary, head and neck, thyroid, parathyroid gland, adrenal gland, bladder, intestine, skin, colorectal, anus, rectum, pancreas, leiomyoma, brain, glioma, glioblastoma, esophagus, liver, kidney, stomach, colon, cervix, uterus, fallopian tubes, endometrium, vulva, larynx, vagina, bone, nasal cavity, paranasal sinus, nasopharynx, oral cavity, oropharynx, larynx, hypolarynx, salivary glands, ureter, urethra, penis and testis.

58. The human anti-PD-1 antibody of any one of claims 1-7, for use in the method of any one of claims 39-46.

59. The human anti-PD-1 antibody of any one of claims 1-7, for use in the method of claim 49.

60. The human anti-PD-1 antibody of any one of claims 1-7, for use in the method of any one of claims 50-54.
